(19) United States
(12) Patent Application Publication
(10) Pub. No.: US 2021/0171653 A1
(43) Pub. Date: Jun. 10, 2021

(54) USE OF ISATUXIMAB FOR THE TREATMENT OF RELAPSED AND/OR REFRACTORY MULTIPLE MYELOMA

(71) Applicants: Sanofi-Aventis U.S. LLC, Bridgewater, NJ (US); Sanofi, Paris (FR)

(72) Inventors: Marie-Laure RISSE, Villejuif (FR); Gaelle ASSET, Ballainvilliers (FR)

(21) Appl. No.: 17/112,862

(22) Filed: Dec. 4, 2020

Related U.S. Application Data
(60) Provisional application No. 63/094,833, filed on Oct. 21, 2020.
(60) Provisional application No. 63/037,353, filed on Jun. 10, 2020.
(60) Provisional application No. 63/023,198, filed on May 11, 2020.
(60) Provisional application No. 62/944,809, filed on Dec. 6, 2019.

(30) Foreign Application Priority Data
Apr. 17, 2020 (EP) 20315186.5

(51) Int. Cl. C07K 16/28; A61P 35/00; A61K 31/573; A61K 31/5355

(52) U.S. Cl. CPC C07K 16/2896; A61P 35/00; A61K 2039/505; A61K 31/5355; A61K 31/573

(57) ABSTRACT

The present disclosure provides methods for treating multiple myeloma (such as refractory multiple myeloma or relapsed and refractory multiple myeloma) in an individual who received one to three prior therapies (or prior lines of therapy) for multiple myeloma. The methods comprise administering to the individual an anti-CD38 antibody, carfilzomib, and dexamethasone.

### CROSS REFERENCE TO RELATED APPLICATIONS

This application claims the priority benefit of U.S. Provisional Application No. 62/944,809, filed Dec. 6, 2019; European Patent Application No. 20315186.5, filed Apr. 17, 2020; U.S. Provisional Application No. 63/023,198, filed May 11, 2020; U.S. Provisional Application No. 63/037,353, filed Jun. 10, 2020; and U.S. Provisional Application No. 63/094,833, filed Oct. 21, 2020; the contents of each of which are incorporated herein by reference in their entirety.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 183952033000SEQLIST.txt, date recorded: Dec. 4, 2020, size: 10 KB).

### FIELD

The present disclosure relates to methods of treating multiple myeloma by administering an anti-CD38 antibody in combination with carfilzomib and dexamethasone.

### BACKGROUND

Multiple myeloma (MM) is a malignant plasma cell disease that is characterized by clonal proliferation of plasma cells in the bone marrow (BM) and the production of excessive amounts of a monoclonal immunoglobulin (usually of the IgG or IgA type or free urinary light chain, i.e., paraprotein, M-protein or M-component). Patients with MM can experience bone pain, bone fractures, fatigue, anemia, infections, hypercalcemia, and kidney problems (Rollig et al. (2015) Lancet. 385(9983):2197-208). The expression of CD38 is especially notable in MM as >98% of patients are positive for this protein (Goldmacher et al. (1994) Blood. 84(9):3017-25; Lin et al. (2004) Am J Clin Pathol. 121(4):482-8). The strong and uniform expression of CD38 on malignant clonal MM cells contrasts with the restricted expression pattern on normal cells suggesting this antigen may be useful for specific targeting of tumor cells.

The current aim of MM therapy is to control the disease as effectively as possible, to maximize quality of life and to prolong survival. The disease trajectory varies for each patient, but relapses are inevitable, and the depth and duration of response to each treatment following relapse are generally diminished. In general, MM patients will receive treatment regimens during their lifespan that include such agents such as proteasome inhibitors (e.g., bortezomib, ixazomib, and carfilzomib) and immune modulatory agents or “IMiDs®” (e.g., lenalidomide, pomalidomide, and thalidomide), monoclonal antibodies (e.g., elotuzumab), histone deacetylase (HDAC) inhibitors (e.g., panobinostat) alone or in combination. However, once a patient becomes refractory to those agents, survival is limited and newer treatment options are needed to treat patients after they have failed stem cell transplant (SCT), chemotherapy, proteasome inhibitors, and immunomodulatory drugs (IMiDs®). Despite the dramatic improvement in patient outcomes with newer therapies, MM remains an incurable disease. Thus, the treatment of patients who have received one to three prior lines of therapy for multiple myeloma remains an unmet medical need.

All references cited herein, including patent applications, patent publications, and UniProtKB/Swiss-Prot Accession numbers are herein incorporated by reference in their entirety, as if each individual reference were specifically and individually indicated to be incorporated by reference.

### SUMMARY

Provided is a method of treating a human individual having multiple myeloma, comprising administering to the individual an anti-CD38 antibody comprising (a) a heavy chain variable domain (V_{H}) that comprises: a CDR-H1 comprising the amino acid sequence DYWMQ (SEQ ID NO: 1), a CDR-H2 comprising the amino acid sequence TIYPGDGDTGYAQKFQG (SEQ ID NO: 2), and a CDR-H3 comprising the amino acid sequence GDYYGSNSLDY (SEQ ID NO: 3), and (b) a light chain variable domain (V_{L}) that comprises: a CDR-L1 comprising the amino acid sequence KASQDVSTVVA (SEQ ID NO: 4), a CDR-L2 comprising the amino acid sequence SASYRYI (SEQ ID NO: 5), and a CDR-L3 comprising the amino acid sequence QQHYSPPYT (SEQ ID NO: 6), carfilzomib, and dexamethasone, wherein the anti-CD38 antibody is administered at a dose of 10 mg/kg, the carfilzomib is administered at a dose of 20 mg/m² or 56 mg/m², and the dexamethasone is administered at a dose of 20 mg, wherein the individual received at least one prior therapy (e.g., one to three prior therapies) for multiple myeloma, and wherein the treatment extends progression free survival (PFS) of the individual. In some embodiments, the treatment extends overall survival (OS) of the individual. Provided is a method of treating a human individual having multiple myeloma, comprising administering to the individual an anti-CD38 antibody comprising (a) a heavy chain variable domain (V_{H}) that comprises: a CDR-H1 comprising the amino acid sequence DYWMQ (SEQ ID NO: 1), a CDR-H2 comprising the amino acid sequence TIYPGDGDTGYAQKFQG (SEQ ID NO: 2), and a CDR-H3 comprising the amino acid sequence GDYYGSNSLDY (SEQ ID NO: 3), and (b) a light chain variable domain (V_{L}) that comprises: a CDR-L1 comprising the amino acid sequence KASQDVSTVVA (SEQ ID NO: 4), a CDR-L2 comprising the amino acid sequence SASYRYI (SEQ ID NO: 5), and a CDR-L3 comprising the amino acid sequence QQHYSPPYT (SEQ ID NO: 6), carfilzomib, and dexamethasone, wherein the anti-CD38 antibody is administered at a dose of 10 mg/kg, the carfilzomib is administered at a dose of 20 mg/m² or 56 mg/m², and the dexamethasone is administered at a dose of 20 mg, wherein the individual received more than three prior therapies for multiple myeloma, and wherein the treatment extends progression free survival (PFS) of the individual. In some embodiments, the treatment extends overall survival (OS) of the individual.

Also provided is a method of treating a human individual having multiple myeloma, comprising administering to the individual an anti-CD38 antibody comprising (a) a heavy chain variable domain (V_{H}) that comprises: a CDR-H1 comprising the amino acid sequence DYWMQ (SEQ ID NO: 1), a CDR-H2 comprising the amino acid sequence TIYPGDGDTGYAQKFQG (SEQ ID NO: 2), and a CDR-H3 comprising the amino acid sequence GDYYGSNSLDY (SEQ ID NO: 3), and (b) a light chain variable domain (V_{L}) that comprises: a CDR-L1 comprising the amino acid sequence KASQDVSTVVA (SEQ ID NO: 4), a CDR-L2 comprising the amino acid sequence SASYRYI (SEQ ID NO: 5), and a CDR-L3 comprising the amino acid sequence QQHYSPPYT (SEQ ID NO: 6), carfilzomib, and dexamethasone, wherein the anti-CD38 antibody is administered at a dose of 10 mg/kg, the carfilzomib is administered at a dose of 20 mg/m² or 56 mg/m², and the dexamethasone is administered at a dose of 20 mg, wherein the individual received at least one prior therapy (e.g., one to three prior therapies) for multiple myeloma, and wherein the treatment extends overall survival (OS) of the individual.

Also provided is a method of treating a human individual having multiple myeloma, comprising administering to the individual an anti-CD38 antibody comprising (a) a heavy chain variable domain (V_{H}) that comprises: a CDR-H1 comprising the amino acid sequence DYWMQ (SEQ ID NO: 1), a CDR-H2 comprising the amino acid sequence TIYPGDGDTGYAQKFQG (SEQ ID NO: 2), and a CDR-H3 comprising the amino acid sequence GDYYGSNSLDY (SEQ ID NO: 3), and (b) a light chain variable domain (V_{L}) that comprises: a CDR-L1 comprising the amino acid sequence KASQDVSTVVA (SEQ ID NO: 4), a CDR-L2 comprising the amino acid sequence SASYRYI (SEQ ID NO: 5), and a CDR-L3 comprising the amino acid sequence QQHYSPPYT (SEQ ID NO: 6), carfilzomib, and dexamethasone, wherein the anti-CD38 antibody is administered at a dose of 10 mg/kg, the carfilzomib is administered at a dose of 20 mg/m² or 56 mg/m², and the dexamethasone is administered at a dose of 20 mg, wherein the individual received more than three prior therapies for multiple myeloma, and wherein the treatment extends overall survival (OS) of the individual.

In some embodiments, provided is a method of treating a human individual having multiple myeloma, comprising administering to the individual an anti-CD38 antibody comprising (a) a heavy chain variable domain (V_{H}) that comprises: a CDR-H1 comprising the amino acid sequence DYWMQ (SEQ ID NO: 1), a CDR-H2 comprising the amino acid sequence TIYPGDGDTGYAQKFQG (SEQ ID NO: 2), and a CDR-H3 comprising the amino acid sequence GDYYGSNSLDY (SEQ ID NO: 3), and (b) a light chain variable domain (V_{L}) that comprises: a CDR-L1 comprising the amino acid sequence KASQDVSTVVA (SEQ ID NO: 4), a CDR-L2 comprising the amino acid sequence SASYRYI (SEQ ID NO: 5), and a CDR-L3 comprising the amino acid sequence QQHYSPPYT (SEQ ID NO: 6), carfilzomib, and dexamethasone, wherein the anti-CD38 antibody is administered at a dose of 10 mg/kg, the carfilzomib is administered at a dose of 20 mg/m² or 56 mg/m², and the dexamethasone is administered at a dose of 20 mg, wherein the individual received at least one prior therapy (e.g., one to three prior therapies) for multiple myeloma, and wherein the individual is Minimal Residual Disease negative at a threshold of 10⁻⁵ or less after treatment.

In some embodiments, provided is a method of treating a human individual having multiple myeloma, comprising administering to the individual an anti-CD38 antibody comprising (a) a heavy chain variable domain (V_{H}) that comprises: a CDR-H1 comprising the amino acid sequence DYWMQ (SEQ ID NO: 1), a CDR-H2 comprising the amino acid sequence TIYPGDGDTGYAQKFQG (SEQ ID NO: 2), and a CDR-H3 comprising the amino acid sequence GDYYGSNSLDY (SEQ ID NO: 3), and (b) a light chain variable domain (V_{L}) that comprises: a CDR-L1 comprising the amino acid sequence KASQDVSTVVA (SEQ ID NO: 4), a CDR-L2 comprising the amino acid sequence SASYRYI (SEQ ID NO: 5), and a CDR-L3 comprising the amino acid sequence QQHYSPPYT (SEQ ID NO: 6), carfilzomib, and dexamethasone, wherein the anti-CD38 antibody is administered at a dose of 10 mg/kg, the carfilzomib is administered at a dose of 20 mg/m² or 56 mg/m², and the dexamethasone is administered at a dose of 20 mg, wherein the individual received at least one prior therapy (e.g., one to three prior therapies) for multiple myeloma, and wherein the individual has renal impairment at the start of treatment.

In some embodiments, the individual received one prior therapy for multiple myeloma. In some embodiments, the individual received more than one prior therapy for multiple myeloma (e.g., such as two prior therapies or three prior therapies). In some embodiments, the individual received more than three prior therapies for multiple myeloma. In some embodiments, the individual received prior therapy with a proteasome inhibitor. In some embodiments, the individual received prior therapy with an immunomodulatory drug (e.g., thalidomide, lenalidomide, and/or pomalidomide). In some embodiments, the individual received prior therapy with a proteasome inhibitor and an immunomodulatory drug. In some embodiments, the individual is classified as Stage I or Stage II according to the Revised International Staging System for multiple myeloma (R-ISS) at the start of treatment. In some embodiments, the individual is classified as Stage III according to R-ISS at the start of treatment. In some embodiments, the individual is not classified according to R-ISS at the start of treatment. In some embodiments, the individual has one or more cytogenetic abnormalities selected from the group consisting of: del(17p), t(4;14), and t(14;16). In some embodiments, the individual has renal impairment at the start of treatment. In some embodiments the individual is 65 to less than 75 years of age at the start of treatment. In some embodiments the individual is 75 years of age or older at the start of treatment.

In some embodiments, the anti-CD38 antibody comprises a heavy chain variable region (V_{H}) comprising an amino acid sequence of SEQ ID NO: 7 and a light chain variable region (V_{L}) comprising an amino acid sequence of SEQ ID NO: 7 or SEQ ID NO: 9. In some embodiments, the anti-CD38 antibody is isatuximab.

In some embodiments, the anti-CD38 antibody, the carfilzomib, and the dexamethasone are administered in a first 28-day cycle, wherein the anti-CD38 antibody is administered at the dose of 10 mg/kg on Days 1, 8, 15, and 22 of the first 28-day cycle, the carfilzomib is administered at the dose of 20 mg/m² on Days 1 and 2 and at a dose of 56 mg/m² on Days 8, 9, 15, and 16 of the first 28-day cycle, and the dexamethasone is administered at the dose 20 mg on Days 1, 2, 8, 9, 15, 16, 22, and 23 of the first 28-day cycle. In some embodiments, the anti-CD38 antibody, the carfilzomib, and the dexamethasone are further administered in one or more 28-day cycles following the first 28-day cycle, wherein the anti-CD38 antibody is administered at the dose of 20 mg/m² on Days 1 and 15 of the one or more 28-day cycles following the first 28-day cycle, the carfilzomib is administered at a dose of 56 mg/m² on each of Days 1, 2, 8, 9, 15, and 16 of the one or more one or more 28-day cycles following the first 28-day cycle, and the dexamethasone is administered at the dose 20 mg on Days 1, 2, 8, 9, 15, 16, 22, and 23 of the one or more one or more 28-day cycles following the first 28-day cycle. In some embodiments, the dexamethasone is administered prior to the anti-CD38 antibody, and wherein the anti-CD38 antibody is administered prior to the carfilzomib on Days 1, 8, and 15 of the first 28 day cycle; and wherein the dexamethasone is administered prior to the anti-CD38 antibody on Day 22 of the first 28-day cycle. In some embodiments, the dexamethasone is administered prior to the anti-CD38 antibody, and wherein the anti-CD38 antibody is administered prior to the carfilzomib on Days 1 and 15 of every 28 day cycle following the first 28 day cycle; and wherein the dexamethasone is administered prior to the carfilzomib on Day 8 of every 28 day cycle following the first 28 day cycle. In some embodiments, the anti-CD38 antibody is administered intravenously. In some embodiments, the carfilzomib is administered intravenously. In some embodiments, the dexamethasone is administered orally.

In some embodiments, the individual is MRD negative at a threshold of 10⁻⁴, 10⁻⁵, 10⁻⁶, or less after treatment.

Also provided herein are kits comprising an anti-CD38 antibody for use in combination with carfilzomib and dexamethasone for treating an individual multiple myeloma according to any one of the methods herein.

Also provided is an anti-CD38 antibody comprising (a) a heavy chain variable domain (V_{H}) that comprises: a CDR-H1 comprising the amino acid sequence DYWMQ (SEQ ID NO: 1), a CDR-H2 comprising the amino acid sequence TIYPGDGDTGYAQKFQG (SEQ ID NO: 2), and a CDR-H3 comprising the amino acid sequence GDYYGSNSLDY (SEQ ID NO: 3), and (b) a light chain variable domain (V_{L}) that comprises: a CDR-L1 comprising the amino acid sequence KASQDVSTVVA (SEQ ID NO: 4), a CDR-L2 comprising the amino acid sequence SASYRYI (SEQ ID NO: 5), and a CDR-L3 comprising the amino acid sequence QQHYSPPYT (SEQ ID NO: 6) for use in a method of treating multiple myeloma in an individual, the method comprising administering to the individual the anti-CD38 antibody, carfilzomib and dexamethasone, wherein the anti-CD38 antibody is administered at a dose of 10 mg/kg, the carfilzomib is administered at a dose of 20 mg/m² or 56 mg/m², and the dexamethasone is administered at a dose of 20 mg, wherein the individual received at least one prior therapy (e.g., one to three prior therapies) for multiple myeloma, and wherein the treatment extends progression free survival (PFS) and/or overall survival (OS) of the individual.

Also provided is an anti-CD38 antibody comprising (a) a heavy chain variable domain (V_{H}) that comprises: a CDR-H1 comprising the amino acid sequence DYWMQ (SEQ ID NO: 1), a CDR-H2 comprising the amino acid sequence TIYPGDGDTGYAQKFQG (SEQ ID NO: 2), and a CDR-H3 comprising the amino acid sequence GDYYGSNSLDY (SEQ ID NO: 3), and (b) a light chain variable domain (V_{L}) that comprises: a CDR-L1 comprising the amino acid sequence KASQDVSTVVA (SEQ ID NO: 4), a CDR-L2 comprising the amino acid sequence SASYRYI (SEQ ID NO: 5), and a CDR-L3 comprising the amino acid sequence QQHYSPPYT (SEQ ID NO: 6) for use in a method of treating multiple myeloma in an individual, the method comprising administering to the individual the anti-CD38 antibody, carfilzomib and dexamethasone, wherein the anti-CD38 antibody is administered at a dose of 10 mg/kg, the carfilzomib is administered at a dose of 20 mg/m² or 56 mg/m², and the dexamethasone is administered at a dose of 20 mg, wherein the individual received more than three prior therapies for multiple myeloma, and wherein the treatment extends progression free survival (PFS) and/or overall survival (OS) of the individual.

In some embodiments, provided is an anti-CD38 antibody comprising (a) a heavy chain variable domain (V_{H}) that comprises: a CDR-H1 comprising the amino acid sequence DYWMQ (SEQ ID NO: 1), a CDR-H2 comprising the amino acid sequence TIYPGDGDTGYAQKFQG (SEQ ID NO: 2), and a CDR-H3 comprising the amino acid sequence GDYYGSNSLDY (SEQ ID NO: 3), and (b) a light chain variable domain (V_{L}) that comprises: a CDR-L1 comprising the amino acid sequence KASQDVSTVVA (SEQ ID NO: 4), a CDR-L2 comprising the amino acid sequence SASYRYI (SEQ ID NO: 5), and a CDR-L3 comprising the amino acid sequence QQHYSPPYT (SEQ ID NO: 6) for use in a method of treating multiple myeloma in an individual, the method comprising the method comprising administering the anti-CD38 antibody, carfilzomib, and dexamethasone to the individual, wherein the anti-CD38 antibody is administered at a dose of 10 mg/kg, the carfilzomib is administered at a dose of 20 mg/m² or 56 mg/m², and the dexamethasone is administered at a dose of 20 mg, wherein the individual received at least one prior therapy for multiple myeloma, and wherein the individual is Minimal Residual Disease negative at a threshold of 10⁻⁵ or less after treatment.

In some embodiments, provided is an anti-CD38 antibody comprising (a) a heavy chain variable domain (V_{H}) that comprises: a CDR-H1 comprising the amino acid sequence DYWMQ (SEQ ID NO: 1), a CDR-H2 comprising the amino acid sequence TIYPGDGDTGYAQKFQG (SEQ ID NO: 2), and a CDR-H3 comprising the amino acid sequence GDYYGSNSLDY (SEQ ID NO: 3), and (b) a light chain variable domain (V_{L}) that comprises: a CDR-L1 comprising the amino acid sequence KASQDVSTVVA (SEQ ID NO: 4), a CDR-L2 comprising the amino acid sequence SASYRYI (SEQ ID NO: 5), and a CDR-L3 comprising the amino acid sequence QQHYSPPYT (SEQ ID NO: 6) for use in a method of treating multiple myeloma in an individual, the method comprising administering the anti-CD38 antibody, carfilzomib and dexamethasone to the individual, wherein the anti-CD38 antibody is administered at a dose of 10 mg/kg, the carfilzomib is administered at a dose of 20 mg/m² or 56 mg/m², and the dexamethasone is administered at a dose of 20 mg, wherein the individual received at least one prior therapy for multiple myeloma, and wherein the individual has renal impairment at the start of treatment.

### BRIEF DESCRIPTION OF THE DRAWINGS

FIG. 1 provides a schematic of the study design of the clinical trial described in the Examples.

FIG. 2 provides an exemplary administration schedule for the anti-CD38 antibody (e.g. isatuximab), carfilzomib, and dexamethasone.

FIG. 3 shows Kaplan-Meier curves of progression-free survival (PFS) for patients receiving isatuximab+carfilzomib+dexamethasone (IKd) vs. patients receiving carfilzomib+dexamethasone (Kd).

FIG. 4 shows a Forest Plot of subgroup analyses for progression-free survival. Circles represent the hazard ratio and the horizontal bars extend from the lower limit to the upper limit of the 95% confidence interval of the estimate of the hazard ratio.

FIG. 5 shows Kaplan-Meier curves of time to next treatment (TNT) for patients receiving isatuximab+carfilzomib+dexamethasone (IKd) vs. patients receiving carfilzomib+dexamethasone (Kd).

FIG. 6 shows a Kapan-Meier curve of progression-free survival by minimal residual disease (MRD) status for patients receiving isatuximab+carfilzomib+dexamethasone (IKd) vs. patients receiving carfilzomib+dexamethasone (Kd).

FIG. 7 shows another Forest Plot of subgroup analyses for progression-free survival. Circles represent the hazard ratio and the horizontal bars extend from the lower limit to the upper limit of the 95% confidence interval of the estimate of the hazard ratio.

### DETAILED DESCRIPTION

### Definitions

As used in this specification and the appended claims, the singular forms “a”, “an” and “the” include plural referents unless the content clearly dictates otherwise. Thus, for example, reference to “a molecule” optionally includes a combination of two or more such molecules, and the like.

“Sustained response” refers to the sustained effect on preventing or delaying progression of a disease (e.g., multiple myeloma) and/or improving one or more response criteria after cessation of a treatment. For example, response to treatment for multiple myeloma may be measured according to the criteria in Kumar et al. (2016) “International Myeloma Working Group consensus criteria for response and minimal residual disease assessment in multiple myeloma.” Lancet Oncol. 17(8): e328-e346) and Durie et al. (2006) “International uniform response criteria for multiple myeloma. Leukemia. 20: 1467-1473. (See also Table A below and Table B herein). In some embodiments, the sustained response has a duration at least the same as the treatment duration, at least 1.5×, 2.0×, 2.5×, or 3.0× length of the treatment duration.

TABLE A
Standard International Myeloma Working Group (IMWG) Response Criteria

| Response | IMWG criteria |
|---|---|
| Complete | Negative immunofixation on the serum and urine and |
| Response | disappearance of any soft tissue plasmacytomas and |
| (CR) | <5% plasma cells in bone marrow aspirates. |
| | Two consecutive assessments are needed. No known evidence of progressive |
| | disease or new bone lesions if radiographic studies were performed |
| Stringent | CR as defined above plus: |
| Complete | Normal free light chain ratio (0.26 to 1.65) and |
| Response | Absence of clonal cells in bone marrow by immunohistochemistry (κ/λ ratio ≤4:1 |
| (sCR) | or ≥1:2 for κ and λ patients, respectively, after counting ≥100 plasma cells). |
| | Two consecutive assessments of laboratory parameters are needed. No known |
| | evidence of progressive disease or new bone lesions if radiographic studies were |
| | performed |
| Very Good | Serum and urine M protein detectable by immunofixation but not on |
| Partial | electrophoresis or |
| Response | ≥90% reduction in serum M protein plus urine M protein level <100 mg/24 hour. |
| (VGPR) | ≥90% decrease in the sum of the products of maximal perpendicular diameter |
| | (SPD) compared to baseline in soft tissue plasmacytoma. |
| | Two consecutive assessments are needed. No known evidence of progressive |
| | disease or new bone lesions if radiographic studies were performed. |
| Partial | ≥50% reduction of serum M protein and reduction in 24 hours urinary M protein |
| Response | by ≥90% or to <200 mg/24 hour |
| (PR) | In addition to the above listed criteria, if present at baseline, a ≥50% reduction in |
| | the size (sum of the products of the maximal perpendicular diameters or “SPD”) of |
| | soft tissue plasmacytomas is also required |
| | Two consecutive assessments are needed. No known evidence of progressive |
| | disease or new bone lesions if radiographic studies were performed. |
| Minimal | ≥25% but ≤49% reduction in serum M protein and reduction in 24 h urine M protein |
| Response | by 50 to 89%, which still exceed 200 mg/24 hour. |
| (MR) | In addition to the above listed criteria, if present at baseline, ≥50% reduction in size |
| | (SPD) of soft tissue plasmacytomas is also required. |
| | Two consecutive assessments are needed. No known evidence of progressive |
| | disease or new bone lesions if radiographic studies were performed. |
| Stable | Not meeting criteria for CR, VGPR, PR, MR or progressive disease. |
| Disease | No known evidence of progressive disease or new bone lesions if radiographic |
| (SD) | studies were performed. |
| Progressive | Any 1 or more of the following criteria: |
| disease | Increase of ≥25% from lowest confirmed value in any 1 of the following criteria: |
| (PD) | Serum M protein (the absolute increase must be ≥0.5 g/dL). |
| | Serum M protein increase ≥1 g/dL if the lowest M component was ≥5 g/dL. |
| | Urine M-component (the absolute increase must be ≥200 mg/24 hour). |
| | Appearance of new lesion(s), ≥50% increase from nadir in SPD of >1 lesion, or |
| | ≥50% increase in the longest diameter of a previous lesion >1 cm in short axis. |
| | Two consecutive assessments are needed for PD on M protein. |

‡SPD, sum of the products of the maximal perpendicular diameters of measured lesions

The term “pharmaceutical formulation” refers to a preparation which is in such form as to permit the biological activity of the active ingredient to be effective, and which contains no additional components which are unacceptably toxic to a subject to which the formulation would be administered. Such formulations are sterile. “Pharmaceutically acceptable” excipients (vehicles, additives) are those that can reasonably be administered to a subject mammal to provide an effective dose of the active ingredient employed.

As used herein, the term “treatment” refers to clinical intervention designed to alter the natural course of the disease or cell (e.g., cancer cell) being treated during the course of clinical pathology. Desirable effects of treatment include decreasing the rate of disease progression, ameliorating or palliating the disease state, and remission or improved prognosis. For example, an individual is successfully “treated” if one or more symptoms associated with cancer are mitigated or eliminated, including, but are not limited to, reducing the proliferation of (or destroying) cancerous cells, decreasing symptoms resulting from the disease, increasing the quality of life of those suffering from the disease, decreasing the dose of other medications required to treat the disease, and/or prolonging survival of individuals.

As used herein, “delaying progression of a disease” means to defer, hinder, slow, retard, stabilize, and/or postpone development of the disease (such as cancer). This delay can be of varying lengths of time, depending on the history of the disease and/or individual being treated. As is evident to one skilled in the art, a sufficient or significant delay can, in effect, encompass prevention, in that the individual does not develop the disease. For example, a late stage cancer, such as development of metastasis, may be delayed.

An “effective amount” is at least the minimum amount required to effect a measurable improvement or prevention of a particular disorder. An effective amount herein may vary according to factors such as the disease state, age, sex, and weight of the individual/patient, and the ability of the antibody to elicit a desired response in the individual. An effective amount is also one in which any toxic or detrimental effects of the treatment are outweighed by the therapeutically beneficial effects. For prophylactic use, beneficial or desired results include results such as eliminating or reducing the risk, lessening the severity, or delaying the onset of the disease, including biochemical, histological and/or behavioral symptoms of the disease, its complications and intermediate pathological phenotypes presenting during development of the disease. For therapeutic use, beneficial or desired results include clinical results such as decreasing one or more symptoms resulting from the disease, increasing the quality of life of those suffering from the disease, decreasing the dose of other medications required to treat the disease, enhancing effect of another medication such as via targeting, delaying the progression of the disease, and/or prolonging survival. In the case of cancer or tumor, an effective amount of the drug may have the effect in reducing the number of cancer cells; reducing the tumor size; inhibiting (i.e., slow to some extent or desirably stop) cancer cell infiltration into peripheral organs; inhibit (i.e., slow to some extent and desirably stop) tumor metastasis; inhibiting to some extent tumor growth; and/or relieving to some extent one or more of the symptoms associated with the disorder. An effective amount can be administered in one or more administrations. For purposes of this invention, an effective amount of drug, compound, or pharmaceutical composition is an amount sufficient to accomplish prophylactic or therapeutic treatment either directly or indirectly. As is understood in the clinical context, an effective amount of a drug, compound, or pharmaceutical composition may or may not be achieved in conjunction with another drug, compound, or pharmaceutical composition. Thus, an “effective amount” may be considered in the context of administering one or more therapeutic agents, and a single agent may be considered to be given in an effective amount if, in conjunction with one or more other agents, a desirable result may be or is achieved.

As used herein, “in conjunction with” refers to administration of one treatment modality in addition to another treatment modality. As such, “in conjunction with” refers to administration of one treatment modality before, during, or after administration of the other treatment modality to the individual.

A “subject” or an “individual” for purposes of treatment refers to any animal classified as a mammal, including humans, domestic and farm animals, and zoo, sports, or pet animals, such as dogs, horses, cats, cows, etc. Preferably, the mammal is human.

The term “antibody” herein is used in the broadest sense and specifically covers monoclonal antibodies (including full length monoclonal antibodies), polyclonal antibodies, multispecific antibodies (e.g., bispecific antibodies), and antibody fragments so long as they exhibit the desired biological activity.

Human light chains are typically classified as kappa and lambda light chains, and human heavy chains are typically classified as mu, delta, gamma, alpha, or epsilon, and define the antibody's isotype as IgM, IgD, IgG, IgA, and IgE, respectively. IgG has several subclasses, including, but not limited to, IgG1, IgG2, IgG3, and IgG4. IgM has subclasses including, but not limited to, IgM1 and IgM2. IgA is similarly subdivided into subclasses including, but not limited to, IgA1 and IgA2. Within full-length light and heavy chains, the variable and constant domains typically are joined by a “J” region of about 12 or more amino acids, with the heavy chain also including a “D” region of about 10 more amino acids. See, e.g., FUNDAMENTAL IMMUNOLOGY (Paul, W., ed., Raven Press, 2nd ed., 1989), which is incorporated by reference in its entirety for all purposes. The variable regions of each light/heavy chain pair typically form an antigen binding site. The variable domains of antibodies typically exhibit the same general structure of relatively conserved framework regions (FR) joined by three hypervariable regions, also called complementarity determining regions or CDRs. The CDRs from the two chains of each pair typically are aligned by the framework regions, which may enable binding to a specific epitope. From the amino-terminus to the carboxyl-terminus, both light and heavy chain variable domains typically comprise, in order, the domains FR1, CDR1, FR2, CDR2, FR3, CDR3, and FR4.

The term “CDR set” refers to a group of three CDRs that occur in a single variable region capable of binding the antigen. The exact boundaries of these CDRs have been defined differently according to different systems. The system described by Kabat (Kabat et al., SEQUENCES OF PROTEINS OF IMMUNOLOGICAL INTEREST (National Institutes of Health, Bethesda, Md. (1987) and (1991)) not only provides an unambiguous residue numbering system applicable to any variable region of an antibody, but also provides precise residue boundaries defining the three CDRs. These CDRs may be referred to as Kabat CDRs.

The term “Fc” as used herein refers to the sequence of a non-antigen-binding fragment that would result from digestion of an antibody or produced by other means, whether in monomeric or multimeric form, and can contain the hinge region. The original immunoglobulin source of the native Fc is preferably of human origin and can be any of the immunoglobulins. Fc molecules are made up of monomeric polypeptides that can be linked into dimeric or multimeric forms by covalent (i.e., disulfide bonds) and non-covalent association. The number of intermolecular disulfide bonds between monomeric subunits of native Fc molecules ranges from 1 to 4 depending on class (e.g., IgG, IgA, and IgE) or subclass (e.g., IgG1, IgG2, IgG3, IgA1, IgGA2, and IgG4). One example of a Fc is a disulfide-bonded dimer resulting from papain digestion of an IgG. The term “native Fc” as used herein is generic to the monomeric, dimeric, and multimeric forms.

As used herein, the term “overall response rate” or “ORR” refers to the proportion of individuals/patients with stringent complete response (sCR), complete response (CR), very good partial response (VGPR), and partial response (PR), as assessed by the IRC using the IMWG response criteria described in Kumar et al. (2016) “International Myeloma Working Group consensus criteria for response and minimal residual disease assessment in multiple myeloma.” Lancet Oncol. 17(8): e328-e346 and Durie et al. (2006) “International uniform response criteria for multiple myeloma. Leukemia. 20: 1467-1473. See also Table A and Table B herein.

### Overview

Provided herein are methods for treating or delaying the progression of multiple myeloma in an individual who has received one, two, three, or more than three prior therapies for multiple myeloma. The methods comprise administering to the individual an effective amount of an anti-CD38 antibody (e.g., isatuximab), carfilzomib, and dexamethasone. In some embodiments, the treatment extends the progression free survival (PFS) and/or the overall survival (OS) of the individual. In some embodiments, the treatment extends the progression free survival (PFS) and/or the overall survival (OS) of the individual, as compared to an individual who is not receiving treatment. In some embodiments, the treatment extends the progression free survival (PFS) and/or the overall survival (OS) of the individual, as compared to an individual receiving treatment with of carfilzomib and dexamethasone, but without the anti-CD38 antibody (e.g., isatuximab). In some embodiments, the individual is negative for minimal residual disease (MRD) (e.g., at a threshold of 10⁻⁴ or less, 10⁻⁵ or less, or 10⁻⁶ or less) after treatment.

### Anti-CD38 Antibodies

In some embodiments, the anti-CD38 antibody binds to human CD38. In some embodiments, the anti-CD38 antibody is a human antibody, a humanized antibody, or a chimeric antibody. In some embodiments, the anti-CD38 antibody comprises (a) a heavy chain variable domain (V_{H}) that comprises: a CDR-H1 comprising the amino acid sequence DYWMQ (SEQ ID NO: 1), a CDR-H2 comprising the amino acid sequence TIYPGDGDTGYAQKFQG (SEQ ID NO: 2), and a CDR-H3 comprising the amino acid sequence GDYYGSNSLDY (SEQ ID NO: 3), and (b) a light chain variable domain (V_{L}) that comprises: a CDR-L1 comprising the amino acid sequence KASQDVSTVVA (SEQ ID NO: 4), a CDR-L2 comprising the amino acid sequence SASYRYI (SEQ ID NO: 5), and a CDR-L3 comprising the amino acid sequence QQHYSPPYT (SEQ ID NO: 6). In some embodiments, the anti-CD38 antibody comprises a heavy chain variable domain (V_{H}) that comprises an amino acid sequence that is at least 90% identical (e.g., at least any one of 91%, 92%, 94%, 95%, 96%, 97%, 98%, or 99%, including any range between these values) to SEQ ID NO: 7. Additionally or alternatively, in some embodiments, the anti-CD38 antibody comprises a light chain variable domain (V_{L}) that comprises an amino acid sequence that is at least 90% identical (e.g., at least any one of 91%, 92%, 94%, 95%, 96%, 97%, 98%, or 99%, including any range between these values) to SEQ ID NO: 8 or SEQ ID NO: 9. In some embodiments, the anti-CD38 antibody comprises a V_{H} that comprises SEQ ID NO: 7 and a V_{L} that comprises SEQ ID NO: 8 or SEQ ID NO: 9.

(SEQ ID NO: 7)
QVQLVQSGAE VAKPGTSVKL SCKASGYTFT DYWMQWVKQR
PGQGLEWIGT IYPGDGDTGY AQKFQGKATL TADKSSKTVY
MHLSSLASED SAVYYCARGD YYGSNSLDYW GQGTSVTVSS
(SEQ ID NO: 8)
DIVMTQSHLS MSTSLGDPVS ITCKASQDVS TVVAWYQQKP
GQSPRRLIYS ASYRYIGVPD RFTGSGAGTD FTFTISSVQA
EDLAVYYCQQ HYSPPYTFGG GTKLEIKR
(SEQ ID NO: 9)
DIVMAQSHLS MSTSLGDPVS ITCKASQDVS TVVAWYQQKP
GQSPRRLIYS ASYRYIGVPD RFTGSGAGTD FTFTISSVQA
EDLAVYYCQQ HYSPPYTFGG GTKLEIKR

In some embodiments, the anti-CD38 antibody is isatuximab (CAS Registry Number: 1461640-62-9). Isatuximab, also known as hu38SB19 and SAR650984, is an anti-CD38 antibody described in WO 2008/047242 and U.S. Pat. No. 8,153,765, the contents of both of which are incorporated by reference herein in their entirety.

The heavy chain of isatuximab comprises the amino acid sequence:

(SEQ ID NO: 10)
QVQLVQSGAE VAKPGTSVKL SCKASGYTFT DYWMQWVKQR
PGQGLEWIGT IYPGDGDTGY AQKFQGKATL TADKSSKTVY
MHLSSLASED SAVYYCARGD YYGSNSLDYW GQGTSVTVSS
ASTKGPSVFP LAPSSKSTSG GTAALGCLVK DYFPEPVTVS
WNSGALTSGV HTFPAVLQSS GLYSLSSVVT VPSSSLGTQT
YICNVNHKPS NTKVDKKVEP KSCDKTHTCP PCPAPELLGG
PSVFLFPPKP KDTLMISRTP EVTCVVVDVS HEDPEVKFNW
YVDGVEVHNA KTKPREEQYN STYRVVSVLT VLHQDWLNGK
EYKCKVSNKA LPAPIEKTIS KAKGQPREPQ VYTLPPSRDE
LIKNQVSLIC LVKGFYPSDI AVEWESNGQP ENNYKTTPPV
LDSDGSFFLY SKLTVDKSRW QQGNVFSCSV MHEALHNHYT
QKSLSLSPG

and the light chain of isatuximab comprises the amino acid sequence:

(SEQ ID NO: 11)
DIVMTQSHLS MSTSLGDPVS ITCKASQDVS TVVAWYQQKP
GQSPRRLIYS ASYRYIGVPD RFTGSGAGTD FTFTISSVQA
EDLAVYYCQQ HYSPPYTFGG GTKLEIKRTV AAPSVFIFPP
SDEQLKSGTA SVVCLLNNFY PREAKVQWKV DNALQSGNSQ
ESVTEQDSKD STYSLSSTLT LSKADYEKHK VYACEVTHQG
LSSPVTKSFN RGEC

The anti-CD38 antibodies may be produced using recombinant methods. For recombinant production of an anti-antigen antibody, nucleic acid encoding the antibody is isolated and inserted into a replicable vector for further cloning (amplification of the DNA) or for expression. DNA encoding the antibody may be readily isolated and sequenced using conventional procedures (e.g., by using oligonucleotide probes that are capable of binding specifically to genes encoding the heavy and light chains of the antibody). Many vectors are available. The vector components generally include, but are not limited to, one or more of the following: a signal sequence, an origin of replication, one or more marker genes, an enhancer element, a promoter, and a transcription termination sequence. The vector is typically transformed into a host cell suitable for expression of the nucleic acid. In some embodiments, the host cell is a eukaryotic cell or a prokaryotic cell. In some embodiments, the eukaryotic host cell is a mammalian cell. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383:44-68 (1982)); MRC 5 cells; FS4 cells; and a human hepatoma line (Hep G2). Other useful mammalian host cell lines include Chinese hamster ovary (CHO) cells, including DHFR-CHO cells (Urlaub et al., Proc. Natl. Acad. Sci. USA 77:4216 (1980)); and myeloma cell lines such as NSO and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, N.J., 2003), pp. 255-268. The anti-CD38 antibody prepared from the cells can be purified using, for example, hydroxylapatite chromatography, hydrophobic interaction chromatography, gel electrophoresis, dialysis, and affinity chromatography, with affinity chromatography being among one of the typically preferred purification steps. In general, various methodologies for preparing antibodies for use in research, testing, and clinical applications are well-established in the art, consistent with the above-described methodologies and/or as deemed appropriate by one skilled in the art.

### Carfilzomib

Carfilzomib is a synthetic tetrapeptide consisting of morpholin-4-acetyl, L-2-amino-4-phenylbutanoyl, L-leucyl and L-phenylalanyl residues joined in sequence with the C-terminus connected to the amino group of (2S)-2-amino-4-methyl-1-[(2R)-2-methyloxiran-2-yl]-1-oxopentan-1-one via an amide linkage. The chemical structure of carfilzomib is shown below:

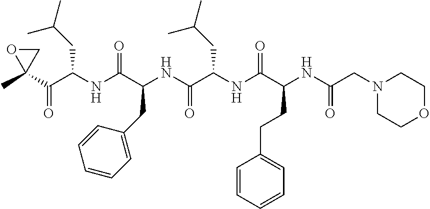 [SMILES: CC(C)C[C@H](NC(=O)[C@H](CCc1ccccc1)NC(=O)CN1CCOCC1)C(=O)N[C@@H](Cc1ccccc1)C(=O)N[C@@H](CC(C)C)C(=O)[C@@]1(C)CO1]

Carfilzomib has molecular formula of C₄₀H₅₇N₅O₇ and a molecular weight of 719.91 g/mol. The CAS Registry Number for carfilzomib is 868540-17-4. Carfilzomib is proteasome inhibitor that is formulated for intravenous administration. Carfilzomib is marketed under the trade name KYPROLIS®.

### Dexamethasone

The chemical name for dexamethasone is 1-Dehydro-16alpha-methyl-9alpha-fluorohydrocortisone, and dexamethasone has the following chemical structure:

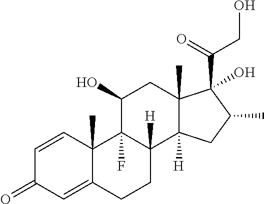

Dexamethasone has molecular formula of C₂₂H₂₉FO₅ and a molecular weight of 392.461 g/mol. Dexamethasone is commercially available as formulations for oral and intravenous administration. Exemplary trade names for dexamethasone include, e.g., DECADRON, MAXIDEX, HEXADROL, DEXACORT, DEXASONE, ORADEXON, SUPERPREDNOL, DEXALONA and others.

### Pharmaceutical Compositions and Formulations

Also provided herein are pharmaceutical compositions and formulations, e.g., for the treatment of multiple myeloma (such as refractory multiple myeloma or relapsed and refractory multiple myeloma) comprising an anti-CD38 antibody (such as isatuximab), carfilzomib, or dexamethasone. In some embodiments, each of the anti-CD38 antibody (e.g., isatuximab), the carfilzomib, and the dexamethasone is provided as a separate pharmaceutical composition. In some embodiments, the pharmaceutical compositions and formulations further comprise a pharmaceutically acceptable carrier.

In some embodiments, an anti-CD38 antibody described herein (such as isatuximab) is in a formulation comprising about 20 mg/mL (500 mg/25 mL) antibody, about 20 mM histidine, about 10% (w/v) sucrose, about 0.02% (w/v) polysorbate 80 at pH 6.0. In some embodiments, an anti-CD38 antibody described herein (such as isatuximab) is in a formulation comprising about 20 mg/mL antibody, about 100 mg/mL sucrose, 2.22 mg/mL histidine hydrochloride monohydrate, about 1.46 mg/ml histidine, and about 0.2 mg/ml polysorbate 80. In some embodiments, the formulation comprises water for injection (WFI), such as sterile water for injection (SWFI). In some embodiments, the formulation is sterile. In some embodiments, a single use of the formulation comprises 5 ml of the formulation (i.e., 100 mg anti-CD38 antibody). In some embodiments, the single use 5 ml formulation is provided in, e.g., a type 16 mL colorless clear glass vial fitted with elastomeric closure. In some embodiments, the fill volume of the vial has been established to ensure removal of 5 mL. In some embodiments, the fill volume is 5.4 mL. In some embodiments, a single use of the formulation comprises 25 ml of the formulation (i.e., 500 mg anti-CD38 antibody). In some embodiments, the single use 25 ml formulation is provided in, e.g., a 30 mL colorless clear glass vial fitted with elastomeric closure. In some embodiments, the fill volume of the vial has been established to ensure removal of 25 mL. In some embodiments, the formulation is stable for at least about 6, 12, 18, 24, 30, or 36 months, including any range in between these values, at a temperature between about 2° C. and about 8° C. and protected from light. In some embodiments, the formulation is diluted for infusion in 0.9% sodium chloride or 5% dextrose. In some embodiments, the diluted infusion solution is stable for up to about 6, 12, 18, 24, 30, 36, 42, or 48 hours, including any range in between these values, between about 2° C. and about 8° C. In some embodiments, the diluted solution for infusion is stable following storage between about 2° C. and about 8° C. for a further 8 hours (including the infusion time) at room temperature. In some embodiments, the diluted solution for infusion is stable in the presence of light. In some embodiments the bag in which the diluted solution for infusion is stored is fabricated from polyolefins (PO), polyethylene (PE), polypropylene (PP), polyvinyl chloride (PVC) with di(ethylhexyl)phthalate (DEHP) or ethyl vinyl acetate (EVA). In some embodiments, the tubing used for infusion is fabricated from PE, PVC (with or without DEHP), polybutyldiene (PBD), or polyurethane (PU) with an in-line filter (polyethersulfone (PES), polysulfone or nylon).

Pharmaceutical formulations of carfilzomib and dexamethasone are commercially available. For example, carfilzomib is known under the trade name KYPROLIS®. Dexamethasone is known under a variety of trade names (as described elsewhere herein), including DECADRON, MAXIDEX, and HEXADROL. In some embodiments, the carfilzomib and/or the dexamethasone are provided in separate containers. In some embodiments, the carfilzomib and/or the dexamethasone are each used and/or prepared for administration to an individual as described in the prescribing information available with the commercially available product.

### Methods of Treatment

Provided herein are methods for treating or delaying progression of multiple myeloma (such as relapsed multiple myeloma or relapsed and refractory multiple myeloma) in an individual (e.g., a human individual) comprising administering to the individual an effective amount of an anti-CD38 antibody (e.g., an anti-CD38 antibody comprising (a) a heavy chain variable domain (V_{H}) that comprises: a CDR-H1 comprising the amino acid sequence DYWMQ (SEQ ID NO: 1), a CDR-H2 comprising the amino acid sequence TIYPGDGDTGYAQKFQG (SEQ ID NO: 2), and a CDR-H3 comprising the amino acid sequence GDYYGSNSLDY (SEQ ID NO: 3), and (b) a light chain variable domain (V_{L}) that comprises: a CDR-L1 comprising the amino acid sequence KASQDVSTVVA (SEQ ID NO: 4), a CDR-L2 comprising the amino acid sequence SASYRYI (SEQ ID NO: 5), and a CDR-L3 comprising the amino acid sequence QQHYSPPYT (SEQ ID NO: 6)), carfilzomib, and dexamethasone, wherein individual received one, two, three, or more than three prior therapies (or lines of therapy) for multiple myeloma. In some embodiments, the individual received no more than three prior therapies (or lines of therapy) In some embodiments, treatment with the anti-CD38 antibody, carfilzomib, and dexamethasone as described herein extends the progression free survival (PFS) of the individual. In some embodiments, treatment with the anti-CD38 antibody, carfilzomib, and dexamethasone as described herein extends the overall survival (OS) of the individual. In some embodiments, treatment with the anti-CD38 antibody, carfilzomib, and dexamethasone as described herein results in lower minimal residual disease (MRD), e.g., as compared to treatment with the carfilzomib and dexamethasone and without the anti-CD38 antibody. In some embodiments the individual is MRD negative following treatment with the anti-CD38 antibody, carfilzomib, and dexamethasone as described herein. In some embodiments, the individual is minimal residual disease (MRD) negative at a threshold of 10⁻⁴ or less after treatment (e.g., wherein “10⁻⁶” means that in a bone marrow sample obtained from the individual after the start of treatment, there is less than 1 tumor cell per 10⁴ bone marrow cells), 10⁻⁵ or less after treatment (e.g., wherein “10⁻⁵” means that in a bone marrow sample obtained from the individual after the start of treatment, there is less than 1 tumor cell per 10⁵ bone marrow cells), or 10⁻⁶ or less after treatment (e.g., wherein “10⁻⁶” means that in a bone marrow sample obtained from the individual after the start of treatment, there is less than 1 tumor cell per 10⁶ bone marrow cells). In some embodiments, MRD is assessed via next generation sequencing (NGS). In some embodiments, MRD is assessed via next generation flow cytometry (NGF). Additionally or alternatively, in some embodiments, MRD is assessed via positron emission tomography-computed tomography (PET-CT) scan. In some embodiments, the individual demonstrates renal impairment prior to treatment (e.g., at the start of treatment) with the anti-CD38 antibody, carfilzomib, and dexamethasone as described herein. In some embodiments, an individual has renal impairment if the individual has a creatine clearance of less than 60 ml/min/1.72 m²(MDRD, or “Modification of Diet in Renal Disease”). In some embodiments, treatment with the anti-CD38 antibody, carfilzomib, and dexamethasone as described herein improves renal function in the individual.

In some embodiments, a treatment is considered a new line of therapy if any one of the following three conditions are met:

1. Start of a new line of treatment after discontinuation of a previous line. If a treatment regimen is discontinued for any reason and a different regimen is started, it can be considered a new line of therapy. For example, a regimen is considered to have been discontinued if all the drugs in that given regimen have been stopped. For example a regimen is not considered to have been discontinued if some of the drugs of the regimen, but not all, have been discontinued. In some embodiments, the reasons for discontinuation, addition, substitution, or stem cell transplantation (SCT) do not influence how lines are counted. Reasons for change may include, for example, end of planned therapy, toxicity, progression, lack of response, inadequate response.

2. The unplanned addition or substitution of 1 or more drugs in an existing regimen. Unplanned addition of a new drug or switching to a different drug (or combination of drugs) due to any reason can be considered a new line of therapy.

3. Stem cell transplantation (SCT). In individuals undergoing >1 SCT, except in the case of a planned tandem SCT with a predefined interval (such as 3 months), each SCT (autologous or allogeneic) can be considered a new line of therapy regardless of whether the conditioning regimen used is the same or different. In some embodiments, planned tandem SCT is considered 1 line. In some embodiments, planned induction and/or consolidation, maintenance with any SCT (frontline, relapse, autologous or allogeneic) is generally considered 1 line of therapy.

In some embodiments, the multiple myeloma is difficult to treat. In some embodiments, the individual has a poor prognosis.

In some embodiments, the individual has multiple myeloma, e.g., relapsed and/or refractory multiple myeloma. In some embodiments, the individual has measurable disease according to one or more of the following criteria: serum M protein ≥0.5 g/dL measured using serum protein immunoelectrophoresis and/or urine M protein ≥200 mg/24 hours measured using urine protein immunoelectrophoresis. In some embodiments, an individual with multiple myeloma (e.g., relapsed and/or refractory multiple myeloma) received at least one, at least two, at least three, or no more than three prior therapies (or lines of therapy) for multiple myeloma. In some embodiments, the individual received prior therapy with a proteasome inhibitor. In some embodiments, the individual received prior therapy with an immunomodulatory drug (e.g., thalidomide, lenalidomide and/or pomalidomide). In some embodiments, the individual received prior therapy with a proteasome inhibitor and an immunomodulatory drug.

In some embodiments, the individual does not have primary refractory multiple myeloma. In some embodiments, an individual with primary refractory multiple myeloma is one who has never achieved at least a minimal response (MR) with any therapy (or line of therapy) during the disease course. In some embodiments, the individual does not have free light chain (FLC) measurable disease only. In some embodiments, the individual has not received prior treatment with an anti-CD38 antibody. In some embodiments, the individual has not received a prior therapy (or a prior line of therapy) with isatuximab. In some embodiments, the individual has not demonstrated progressive disease (PD) during a prior therapy (or prior line of therapy) with an anti-CD38 antibody. In some embodiments, the individual has not demonstrated progression within 60 days after the end of a therapy (or line of therapy) with an anti-CD38 antibody. In some embodiments, the individual has not failed to achieve at least minimal response to therapy (or line of therapy) comprising an anti-CD38 antibody. In some embodiments, the individual who has received prior therapy (or a line of therapy) comprising an anti-CD38 antibody was not refractory to the anti-CD38 antibody. In some embodiments, the individual has not received prior treatment with carfilzomib. In some embodiments, the individual is not allergic to (or has no known allergy to) CAPTISOL® (a cyclodextrin derivative used to solubilize carfilzomib). In some embodiments, the individual is not hypersensitive to or has not demonstrated hypersensitivity to sucrose, histidine (as base and hydrochloride salt), polysorbate 80, or any of the components (active substance or excipient) of the anti-CD38 antibody, carfilzomib, and dexamethasone that are not amenable to premedication with steroids, or H2 blockers, that would prohibit further treatment with these agents. In some embodiments, the individual is not contraindicated for dexamethasone. In some embodiments, the individual has not undergone prior allogenic hematopoietic stem cell transplant with active graft versus host disease (any grade and/or being under immunosuppressive treatment within 2 months before the start of treatment). In some embodiments, the individual does not have known amyloidosis or concomitant plasma cell leukemia. In some embodiments, the individual does not have pleural effusions requiring thoracentesis or ascites requiring paracentesis or any major procedures, e.g., plasmapheresis, curative radiotherapy, major surgery (not including kyphoplasty). In some embodiments, the individual does not have an Eastern Cooperative Oncology Group (ECOG) performance status (PS) ≥2. In some embodiments, the individual does not have platelets <50,000 cells/4 if <50% of bone marrow (BM) nucleated cells are plasma cells and <30,000 cells/4 if ≥50% of BM nucleated cells are plasma cells. In some embodiments, the individual does not have absolute neutrophil count (ANC)<1000 μ/L (1×109/L). In some embodiments, the individual does not have creatinine clearance <15 mL/min/1.73 m² (Modification of Diet in Renal Disease [MDRD] Formula). In some embodiments, the individual does not have total bilirubin >1.5× upper limit of normal (ULN), except for known Gilbert syndrome. In some embodiments, the individual does not have corrected serum calcium >14 mg/dL (>3.5 mmol/L). In some embodiments, the individual does not have aspartate aminotransferase (AST) and/or alanine aminotransferase (ALT) >3×ULN. In some embodiments, the individual does not have ongoing toxicity (excluding alopecia and those listed in the paragraph above) from any prior anti-myeloma therapy of Grade >1 (National Cancer Institute Common Toxicity Criteria for Adverse Events [NCI-CTCAE] v4.03). In some embodiments, the individual does not have prior malignancy. In some embodiments, adequately treated basal cell or squamous cell skin or superficial (pTis, pTa, and pT1) bladder cancer or low risk prostate cancer or any in situ malignancy after curative therapy, as well as any other cancer for which therapy has been completed ≥5 years prior to start of treatment with the anti-CD38 antibody, carfilzomib, and dexamethasone, and from which the individual has been disease free for ≥5 years is not considered a prior malignancy. In some embodiments, the individual has not had myocardial infarction, severe/unstable angina pectoris, coronary/peripheral artery bypass graft, New York Heart Association class III or IV congestive heart failure, Grade ≥3 arrhythmias, stroke, or transient ischemic attack. In some embodiments, the individual has not had myocardial infarction, severe/unstable angina pectoris, coronary/peripheral artery bypass graft, New York Heart Association class III or IV congestive heart failure, Grade ≥3 arrhythmias, stroke, or transient ischemic attack within six months of the start of treatment with the anti-CD38 antibody, carfilzomib, and dexamethasone. In some embodiments, the individual does not have Left ventricular ejection fraction (LVEF) <40%. In some embodiments, the individual does not have or is not known to have acquired immunodeficiency syndrome (AIDS) related illnesses or HIV disease requiring antiretroviral treatment, or to have active hepatitis A, B (defined as a known positive hepatitis B surface antigen (HBsAg) result), or C (defined as a known quantitative hepatitis C (HCV) ribonucleic acid (RNA) results greater than the lower limits of detection of the assay or positive HCV antigen) infection. In some embodiments, the individual does not have any of the following within 3 months prior to the start of treatment with the anti-CD38 antibody, carfilzomib, and dexamethasone: treatment resistant peptic ulcer disease, erosive esophagitis or gastritis, infectious or inflammatory bowel disease, diverticulitis, pulmonary embolism, or other uncontrolled thromboembolic event.

In some embodiments, the treatment comprises administering the anti-CD38 antibody, the carfilzomib, and the dexamethasone in 28-day cycles (e.g., one or more 28-day cycles).

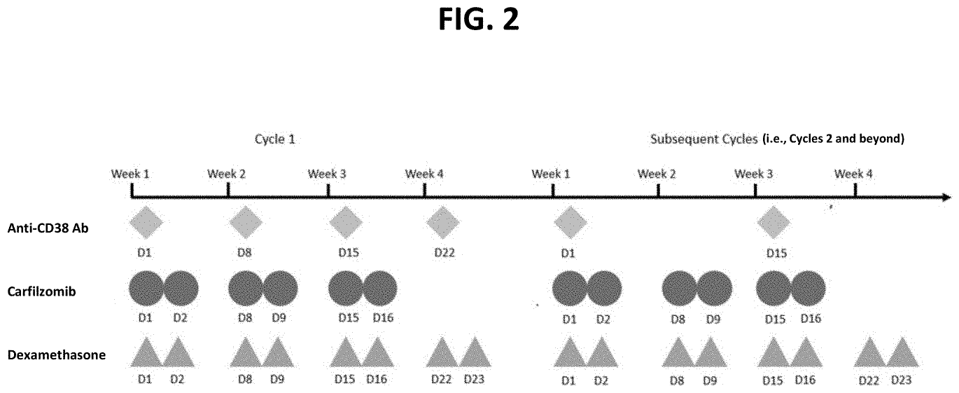

In some embodiments, treatment comprises administering the anti-CD38 antibody, the carfilzomib, and the dexamethasone in a first 28-day cycle (i.e., Cycle 1), wherein the anti-CD38 antibody (e.g., isatuximab) is administered on Days 1, 8, 15, and 22; the carfilzomib is administered on Days 1, 2, 8, 9, 15 and 16; and the dexamethasone is administered on Days 1, 2, 8, 9, 15, 16, 22, and 23. See, e.g., FIG. 2. In some embodiments, treatment comprises administering the anti-CD38 antibody, the carfilzomib, and the dexamethasone in one or more additional 28-day cycles after the first 28-day cycle (e.g., Cycle 2 and beyond), wherein the anti-CD38 antibody (e.g., isatuximab) is administered on Days 1 and 15; the carfilzomib is administered on Days 1, 2, 8, 9, 15 and 16; and the dexamethasone is administered on Days 1, 2, 8, 9, 15, 16, 22, and 23. See, e.g., FIG. 2.

In some embodiments, treatment comprises administering the anti-CD38 antibody, the carfilzomib, and the dexamethasone in a first 28-day cycle (i.e., Cycle 1), wherein the anti-CD38 antibody (e.g., isatuximab) is administered at a dose of 10 mg/kg on Days 1, 8, 15, and 22; the carfilzomib is administered at a dose of 20 mg/m² on Days 1 and 2 and at a dose of 56 mg/m² on Days 8, 9, 15 and 16; and the dexamethasone is administered at a dose of 20 mg on Days 1, 2, 8, 9, 15, 16, 22, and 23. See, e.g., Table D herein. In some embodiments, treatment comprises administering the anti-CD38 antibody, the carfilzomib, and the dexamethasone in one or more additional 28-day cycles after the first 28-day cycle (e.g., Cycle 2 and beyond), wherein the anti-CD38 antibody (e.g., isatuximab) is administered at a dose of 10 mg/kg on Days 1 and 15; the carfilzomib is administered at a dose of 56 mg/m² on Days 1, 2, 8, 9, 15 and 16; and the dexamethasone is administered at a dose of 20 mg on Days 1, 2, 8, 9, 15, 16, 22, and 23. See, e.g., Table D herein.

In some embodiments, the anti-CD38 antibody (e.g., isatuximab), the carfilzomib, and the dexamethasone are administered simultaneously. In some embodiments, the anti-CD38 antibody (e.g., isatuximab), the carfilzomib, and the dexamethasone are administered concurrently. In some embodiments, the anti-CD38 antibody (e.g., isatuximab), the carfilzomib, and the dexamethasone are administered sequentially. In some embodiments, wherein the anti-CD38 antibody (e.g., isatuximab), the carfilzomib, and the dexamethasone are administered sequentially, the dexamethasone is administered prior to the anti-CD38 antibody, and the anti-CD38 antibody administered prior to the carfilzomib on the days of each 28-day cycle where all three of the anti-CD38 antibody, the carfilzomib, and the dexamethasone are administered. In some embodiments, wherein the anti-CD38 antibody (e.g., isatuximab), the carfilzomib, and the dexamethasone are administered sequentially, the dexamethasone is administered prior to the carfilzomib on the days of each 28-day cycles where there is no anti-CD38 administration. In some embodiments, the anti-CD38 antibody (such as isatuximab) is administered intravenously. In some embodiments, the carfilzomib is administered intravenously. In some embodiments, the dexamethasone is administered intravenously, or orally. In some embodiments, the dexamethasone is administered intravenously on the days of each 28-day cycle

In some embodiments, the PFS of the individual is measured as the period of time from the start of treatment to the first occurrence of progressive disease (PD). In some embodiments, PD is assessed according to the criteria in Kumar et al. (2016) “International Myeloma Working Group consensus criteria for response and minimal residual disease assessment in multiple myeloma.” Lancet Oncol. 17(8): e328-e346) and Durie et al. (2006) “International uniform response criteria for multiple myeloma. Leukemia. 20: 1467-1473. (See also Table A and Table B). In some embodiments, PFS is measured as the time from the start of treatment to the time of death. In some embodiments, the methods and uses provided herein result in improved (e.g., extend) progression free survival (PFS) of the individual, as compared to an individual having multiple myeloma (such as refractory multiple myeloma or relapsed and refractory multiple myeloma) who received treatment comprising carfilzomib and dexamethasone without the anti-CD38 antibody. In some embodiments, the treatment increases the PFS of the individual.

In some embodiments, overall survival (OS) is measured as the period of time from the start of treatment to death. In some embodiments, the treatment increases the OS of the individual as compared to an individual having multiple myeloma (such as refractory multiple myeloma or relapsed and refractory multiple myeloma) who received treatment comprising carfilzomib and dexamethasone without the anti-CD38 antibody.

In some embodiments, the time to first response in an individual receiving treatment with the anti-CD38 antibody, carfilzomib, and dexamethasone is shorter than the time to first response in individual receiving treatment with carfilzomib and dexamethasone. In some embodiments, “time to first response” refers to the duration of time between the date of the first dose and the date of the first sign of response (see, e.g., Table A). In some embodiments, the duration of response (DOR) of an individual receiving treatment with the anti-CD38 antibody, carfilzomib, and dexamethasone is longer than the DOR of individual receiving treatment with carfilzomib and dexamethasone. In some embodiments, DOR refers to the time from the date of response for an individual (or individuals) achieving partial response (PR) or better to the date of first documented progressive disease (PD) or death, whichever happens first.

In some embodiments, the individual is negative for minimal residual disease (MRD) or “MRD-negative” following treatment with the anti-CD38 antibody, carfilzomib, and dexamethasone. In some embodiments, MRD status is measured by next generation flow cytometry (NGF). In some embodiments, MRD-negative as measured by NGF (or “flow MRD-negative”) refers to the absence of phenotypically aberrant clonal plasma cells (such as multiple myeloma cells) in bone marrow aspirates (for example using the EUROFLOW™ high-throughput flow cytometry standard operation procedure for MRD detection in multiple myeloma (see Flores-Montero et al. (2017) Leukemia. 31: 2094-2103) or an equivalent method) with a minimum sensitivity of, e.g., 1 in 10⁴ nucleated cells (or “10⁻⁴”), 1 in 10⁵ nucleated cells (or “10⁻⁵”), 1 in 10⁶ nucleated cells (or “10⁻⁶”), or 1 in 10⁷ nucleated cells (or “10⁻⁷”). In some embodiments, MRD status is measured by next generation sequencing (NGS). In some embodiments, MRD-negative as measured by NGS (or “sequencing MRD negative”) refers to absence of clonal plasma cells (e.g., multiple myeloma cells) in bone marrow aspirates; the presence of a clone is defined as at least two identical sequencing reads obtained after DNA sequencing of bone marrow aspirates (for example, using the LYMPHOSIGHT® high-throughput sequencing platform or equivalent method) with a minimum sensitivity of, e.g., 1 in 10⁴ nucleated cells(or “10⁻⁴”), 1 in 10⁵ nucleated cells (or “10⁻⁵”), 1 in 10⁶ nucleated cells (or “10⁻⁶”), or higher. In some embodiments, the minimum sensitivity is 1 cell in 10⁶ nucleated cells (“10⁻⁶”). In some embodiments, the individual is negative by both imaging and MRD (or “imaging+MRD negative”). In some embodiments, imaging+MRD negative refers to (a) being MRD-negative as detected by NGF or MRD-negative as detected by NGS and (b) disappearance of every area of increased tracer uptake found at baseline or a preceding positron emission tomography (PET)/computed tomography (Ct) or decrease to <mediastinal blood pool maximum standardized uptake value or decrease to less than that of surrounding normal tissue. In some embodiments, the individual is “sustained MRD-negative.” In some embodiments, sustained MRD negativity refers to an individual who has been confirmed to be imaging+MRD-negative at two time points following the start of treatment, wherein the time points are no less than 1 year apart. In some embodiments, minimal residual disease (MRD) is assessed via NGF or NGS using a bone marrow sample collected from an individual who has received treatment with the anti-CD38 antibody (e.g., isatuximab), carfilzomib, and dexamethasone, as described herein. In some embodiments, the individual who is assessed for MRD has achieved complete response or better (i.e., ≥CR), or has achieved very good partial response or better (i.e., ≥VGPR) during or after treatment with the anti-CD38 antibody (e.g., isatuximab), carfilzomib, and dexamethasone, as described herein. In some embodiments, the individual treated with the anti-CD38 antibody (e.g., isatuximab), carfilzomib, and dexamethasone who achieves MRD-negative status has renal impairment, e.g., an eGFR <60 mL/min/1.73 m², at the start of treatment, during treatment, or after treatment. In some embodiments, the individual treated with the anti-CD38 antibody (e.g., isatuximab), carfilzomib, and dexamethasone who achieves MRD-negative status is classified as ISS stage III at diagnosis. In some embodiments, the individual treated with the anti-CD38 antibody (e.g., isatuximab), carfilzomib, and dexamethasone who achieves MRD-negative status has one or more cytogenetic abnormalities selected from: t(4;14) and gain(1q21). In some embodiments, the individual treated with the anti-CD38 antibody (e.g., isatuximab), carfilzomib, and dexamethasone who achieves MRD-negative status is heavily pretreated, e.g., has received ≥3 prior lines of therapy for multiple myeloma. In some embodiments, the individual treated with the anti-CD38 antibody (e.g., isatuximab), carfilzomib, and dexamethasone who achieves MRD-negative status was refractory to lenalidomide in their last their regimen (e.g., last treatment regimen for multiple myeloma).

In some embodiments of any of the methods of anti-CD38 antibodies for use described herein, the individual is less than 65 years of age. In some embodiments, the individual is between 65 and less than 75 years of age. In some embodiments, the individual is 75 years of age or older. In some embodiments, the individual is female (e.g. a fertile female of childbearing age). In some embodiments, where the individual is female and is able to become pregnant, the individual may use an effective method of contraception during the treatment with the anti-CD38 antibody and for five months after the last dose of the anti-CD38 antibody.

In some embodiments, the individual has undergone one prior therapy (or prior line of therapy) for multiple myeloma. In some embodiments, the individual has undergone more than one (e.g., two, three, or more than three) prior therapies (or prior lines of therapy) for multiple myeloma. In some embodiments, the individual has undergone more than one but no more than three prior therapies (or prior lines of therapy) for multiple myeloma. In some embodiments, the individual has undergone more than three prior therapies (or prior lines of therapy) for multiple myeloma. In some embodiments, the individual is Stage I or Stage II according to the Multiple Myeloma Revised International Stating System (R-ISS). In some embodiments, Stage I according to the Multiple Myeloma R-ISS is defined as (a) serum beta-2 microglobulin level less than 3.5 mg/L, (b) serum albumin greater than or equal to 3.5 g/dL, (c) standard risk chromosomal/cytogenetic abnormalities detected by interphase fluorescent in situ hybridization (iFISH) and (d) a normal serum lactate dehydrogenase (LDH) level. In some embodiments, Stage II according to the Multiple Myeloma R-ISS is defined as not R-ISS Stage I or Stage III. In some embodiments, the individual is Stage III according to the Multiple Myeloma Revised International Stating System (R-ISS). In some embodiments, Stage III according to the Multiple Myeloma R-ISS is defined as (a) a serum beta-2 microglobulin level of greater than about 5.5 mg/L and either (b) high-risk cytogenetic abnormality detected by interphase fluorescent in situ hybridization (iFISH) or (c) a serum lactate dehydrogenase (LDH) level greater than the upper limit of normal. In some embodiments, the individual has a high-risk cytogenetic abnormality (CA). In some embodiments, the high-risk cytogenetic abnormality is one or more of del(17p), t(4:14), and/or t(14;16). In some embodiments, the individual is not classified according to the R-ISS. In some embodiments, the individual is not classified according to the R-ISS due to inconclusive iFISH.

In some embodiments, the individual has one or more high-risk cytogenetic abnormalities selected from del(17p), t(4:14), and t(14:16). Additionally or alternatively, in some embodiments, the individual has a del(1p), the gain (1q), or both the del(1p) and gain (1q) cytogenetic abnormalities.

### Intravenous Administration of Anti-CD38 Antibody

In some embodiments, the anti-CD38 antibody is administered via intravenous infusion, wherein each infusion is from a volume (e.g., a fixed volume) of 250 ml. In some embodiments, the individual does not experience an infusion reaction (IR) during or following the administration of the anti-CD38 antibody via intravenous infusion from the 250 ml volume. In some embodiments, the individual experiences only mild IR during or following the administration of the anti-CD38 antibody via intravenous infusion from the 250 ml volume.

In some embodiments, the anti-CD38 antibody (e.g., isatuximab) is administered to the individual in a first 28-day cycle. In some embodiments, the anti-CD38 antibody (e.g., isatuximab) is administered to the individual at a dose of 10 mg/kg from a volume of 250 ml on each of Days 1, 8, 15, and 22 of the first 28-day cycle. In some embodiments, the anti-CD38 antibody (e.g., isatuximab) is administered to the individual via intravenous infusion on Day 1 of the first 28 day cycle at an infusion rate of 25 mL/hour for a first hour, and the infusion rate is increased by 25 mL/hour every 30 minutes after the first hour to a maximum infusion rate of 150 mL/hour until the 250 ml of the anti-CD38 antibody (e.g., isatuximab) is infused. In some embodiments, the anti-CD38 antibody (e.g., isatuximab) is administered to the individual via intravenous infusion on Day 1 of the first 28 day cycle at an infusion rate of 12.5 mL/hour for a first 30 minutes, wherein the infusion rate is increased by 25 mL/hour every 30 minutes after the first 30 minutes until the 250 ml of the anti-CD38 antibody (e.g., isatuximab) is infused. In some embodiments, the duration of the infusion of the anti-CD38 antibody (e.g., isatuximab) on Day 1 of the first 28-day cycle is no more than any one of about 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8. 4.9, 5.0, 5.1, 5.2, 5.3, 5.4, 5.5, 5.6, 5.7, 5.8, 5.9, 6.0, 6.2, 6.3, 6.4, or 6.5 hours, including any range in between these values. In some embodiments, the duration of the infusion of the anti-CD38 antibody (e.g., isatuximab) on Day 1 of the first 28-day cycle is between about 3.3 and about 6.1 hours, including any value within in this range. In some embodiments, the duration of the infusion of the anti-CD38 antibody (e.g., isatuximab) on Day 1 of the first 28-day cycle is between about 3.2 and 5.5 hours, such as between about 3.36 and about 5.32 hours. In some embodiments, the duration of the infusion of the anti-CD38 antibody (e.g., isatuximab) on Day 1 of the first 28-day cycle is between about 3.8 and 4.2 hours, such as about 3.94 hours. In some embodiments, the duration of infusion includes temporary interruptions prior to completion of the infusion.

In some embodiments, the anti-CD38 antibody (e.g., isatuximab) is administered to the individual via intravenous infusion on Day 8 of the first 28 day cycle at an infusion rate of 50 mL/hour for a first 30 minutes, 100 mL/hour for a second 30 minutes, 200 mL for the third 30 minutes, and 300 mL/hour after the third 30 minutes until the 250 ml of the anti-CD38 antibody (e.g., isatuximab) is infused. In some embodiments, the anti-CD38 antibody (e.g., isatuximab) is administered to the individual via intravenous infusion on Day 8 of the first 28 day cycle at an infusion rate of 25 mL/hour for a first 30 minutes, and the infusion rate is increased by 50 mL/hour every 30 minutes after the first 30 minutes until the 250 ml of the anti-CD38 antibody (e.g., isatuximab) is infused. In some embodiments, the duration of the infusion of the anti-CD38 antibody (e.g., isatuximab) on Day 1 of the first 28-day cycle is no more than any one of about 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, or 4.0 hours, including any range in between these values. In some embodiments, the duration of the infusion of the anti-CD38 antibody (e.g., isatuximab) on Day 8 of the first 28-day cycle is between about 1.5 and about 3.5 hours, including any value within in this range. In some embodiments, the duration of the infusion of the anti-CD38 antibody (e.g., isatuximab) on Day 8 of the first 28-day cycle is between about 1.4 and 2.7 hours, such as between about 1.52 and about 2.6 hours. In some embodiments, the duration of the infusion of the anti-CD38 antibody (e.g., isatuximab) on Day 8 of the first 28-day cycle is between about 1.5 and 2.0 hours, such as about 1.88 hours. In some embodiments, the duration of infusion of the anti-CD38 antibody (e.g., isatuximab) includes temporary interruptions prior to completion of the infusion.

In some embodiments, the anti-CD38 antibody (e.g., isatuximab) is administered to the individual via intravenous infusion on Day 15 of the first 28-day cycle at an infusion rate of 200 ml/hour until the 250 ml of the anti-CD38 antibody (e.g., isatuximab) is infused. In some embodiments, the anti-CD38 antibody (e.g., isatuximab) is administered to the individual via intravenous infusion on Day 15 of the first 28 day cycle at an infusion rate of 100 ml/hour for a first 30 minutes, and the infusion rate is increased by 50 mL/hour every 30 minutes after the first 30 minutes until the 250 ml of the anti-CD38 antibody (e.g., isatuximab) is infused. In some embodiments, the duration of the infusion of the anti-CD38 antibody (e.g., isatuximab) on Day 15 of the first 28-day cycle is no more than any one of about 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, or 4.0 hours, including any range in between these values. In some embodiments, the duration of the infusion of the anti-CD38 antibody (e.g., isatuximab) on Day 1 of the first 28-day cycle is between about 1.2 and about 3.4 hours, including any value within in this range. In some embodiments, the duration of the infusion the anti-CD38 antibody (e.g., isatuximab) on Day 15 of the first 28-day cycle is between about 1 and 2 hours, such as between about 1.03 and about 1.87 hours. In some embodiments, the duration of the infusion on Day 15 of the first 28-day cycle is between about 1 and 1.5 hours, such as about 1.27 hours. In some embodiments, the duration of infusion of the anti-CD38 antibody (e.g., isatuximab) includes temporary interruptions prior to completion of the infusion.

In some embodiments, the anti-CD38 antibody (e.g., isatuximab) is administered to the individual via intravenous infusion on Day 22 of the first 28-day cycle at an infusion rate of 200 ml/hour until the 250 ml of the anti-CD38 antibody (e.g., isatuximab) is infused. In some embodiments, the anti-CD38 antibody (e.g., isatuximab) is administered to the individual via intravenous infusion on Day 22 of the first 28 day cycle at an infusion rate of 100 ml/hour for a first 30 minutes, and wherein the infusion rate is increased by 50 mL/hour every 30 minutes after the first 30 minutes until the 250 ml of the anti-CD38 antibody (e.g., isatuximab) is infused. In some embodiments, the duration of the infusion of the anti-CD38 antibody (e.g., isatuximab) on Day 22 of the first 28-day cycle is no more than any one of about 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, or 4.0 hours, including any range in between these values. In some embodiments, the duration of the infusion of the anti-CD38 antibody (e.g., isatuximab) on Day 22 of the first 28-day cycle is between about 1.1 and about 2 hours, including any value within in this range. In some embodiments, the duration of the infusion of the anti-CD38 antibody (e.g., isatuximab) on Day 22 of the first 28-day cycle is between about 1 and 2 hours, such as between about 1.18 and about 1.52 hours. In some embodiments, the duration of the infusion of the anti-CD38 antibody (e.g., isatuximab) on Day 22 of the first 28-day cycle is between about 1 and 1.5 hours, such as about 1.27 hours. In some embodiments, the duration of infusion of the anti-CD38 antibody (e.g., isatuximab) includes temporary interruptions prior to completion of the infusion.

In some embodiments, the anti-CD38 antibody (e.g., isatuximab) is further administered in one or more subsequent 28-day cycles (e.g., following the first 28-day cycle) at a dose of 10 mg/kg from a volume of 250 ml on each of Days 1 and 15 of each subsequent 28-day cycle. In some embodiments, the anti-CD38 antibody (e.g., isatuximab) is administered to the individual via intravenous infusion on Day 1 of each subsequent 28-day cycle (e.g., following the first 28-day cycle) at an infusion rate of 200 ml/hour until the 250 ml of the anti-CD38 antibody (e.g., isatuximab) is infused. In some embodiments, the anti-CD38 antibody (e.g., isatuximab) is administered to the individual via intravenous infusion on Day 1 of each subsequent 28 day cycle (e.g., following the first 28-day cycle) at an infusion rate of 100 ml/hour for a first 30 minutes, and wherein the infusion rate is increased by 50 mL/hour every 30 minutes after the first 30 minutes until the 250 ml of the anti-CD38 antibody (e.g., isatuximab) is infused. In some embodiments, the duration of the infusion of the anti-CD38 antibody (e.g., isatuximab) on Day 1 of each subsequent 28 day cycle (e.g., following the first 28-day cycle) is no more than any one of about 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, or 4.0 hours, including any range in between these values. In some embodiments, the duration of the infusion of the anti-CD38 antibody (e.g., isatuximab) on Day 1 of each subsequent 28-day cycle (e.g., following the first 28-day cycle) is between about 1.1 and about 1.6 hours, including any value within in this range. In some embodiments, the duration of the infusion of the anti-CD38 antibody (e.g., isatuximab) on Day 1 of each subsequent 28-day cycle (e.g., following the first 28-day cycle) is between about 1 and 2 hours, such as between about 1.19 and about 1.41 hours. In some embodiments, the duration of the infusion of the anti-CD38 antibody (e.g., isatuximab) on Day 1 of each subsequent 28-day cycle (e.g., following the first 28-day cycle) is between about 1 and 1.5 hours, such as about 1.27 hours. In some embodiments, the duration of infusion of the anti-CD38 antibody (e.g., isatuximab) includes temporary interruptions prior to completion of the infusion. In some embodiments, the anti-CD38 antibody (e.g., isatuximab) is administered to the individual via intravenous infusion on Day 15 of each subsequent 28-day cycle (e.g., following the first 28-day cycle) at an infusion rate of 200 ml/hour until the 250 ml of the anti-CD38 antibody (e.g., isatuximab) is infused. In some embodiments, the anti-CD38 antibody (e.g., isatuximab) is administered to the individual via intravenous infusion on Day 15 of each subsequent 28 day cycle (e.g., following the first 28 day cycle) at an infusion rate of 100 ml/hour for a first 30 minutes, and wherein the infusion rate is increased by 50 mL/hour every 30 minutes after the first 30 minutes until the 250 ml of the anti-CD38 antibody (e.g., isatuximab) is infused. In some embodiments, the duration of the infusion of the anti-CD38 antibody (e.g., isatuximab) on Day 15 of each subsequent 28 day cycle (e.g., following the first 28-day cycle) is no more than any one of about 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, or 4.0 hours, including any range in between these values. In some embodiments, the duration of the infusion of the anti-CD38 antibody (e.g., isatuximab) on Day 1 of each subsequent 28-day cycle (e.g., following the first 28-day cycle) is between about 1.2 and about 1.6 hours, including any value within in this range. In some embodiments, the duration of the infusion of the anti-CD38 antibody (e.g., isatuximab) on Day 1 of each subsequent 28-day cycle (e.g., following the first 28-day cycle) is between about 1 and 2 hours, such as between about 1.2 and about 1.46 hours. In some embodiments, the duration of the infusion of the anti-CD38 antibody (e.g., isatuximab) on Day 1 of each subsequent 28-day cycle (e.g., following the first 28-day cycle) is between about 1 and 1.5 hours, such as about 1.27 hours. In some embodiments, the duration of infusion of the anti-CD38 antibody (e.g., isatuximab) includes temporary interruptions prior to completion of the infusion.

In some embodiments, the duration of each infusion of the anti-CD38 antibody (e.g., isatuximab) on or after Day 15 of the first 28 day cycle (e.g., including Day 22 of the first 28 day cycle and Day 1 and Day 15 of each subsequent 28-day cycle) is no more than any one of about 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, or 4.0 hours, including any range in between these values. In some embodiments, the duration of each infusion of the anti-CD38 antibody (e.g., isatuximab) on or after Day 15 of the first 28-day cycle (e.g., including Day 22 of the first 28-day cycle and Day 1 and Day 15 of each subsequent 28-day cycle) is between about 0.7 and about 3.4 hours, including any value within in this range. In some embodiments, the duration of each infusion of the anti-CD38 antibody (e.g., isatuximab) on or after Day 15 of the first 28 day cycle (e.g., including Day 22 of the first 28 day cycle and Day 1 and Day 15 of each subsequent 28-day cycle) is between about 1 and 2 hours, such as between about 1.13 and about 1.53 hours. In some embodiments, the duration of the infusion of the anti-CD38 antibody (e.g., isatuximab) on Day 1 of each subsequent 28-day cycle (e.g., following the first 28-day cycle) is between about 1 and 1.5 hours, such as about 1.25 hours.

In some embodiments, the individual does not experience an infusion reaction (IR) during or following administration (e.g., intravenous infusion) of the anti-CD38 antibody (such as isatuximab) at a dose of 10 mg/kg from a 250 ml volume. In some embodiments, administration of the anti-CD38 antibody (e.g., via intravenous infusion) at a dose of 10 mg/kg from a 250 ml volume does not cause the individual to experience an IR during or following administration. An IR refers to a disorder characterized by adverse reaction to the intravenous infusion of an anti-CD38 antibody (e.g., isatuximab). An IR may occur during the fusion or within 24 hours of the infusion (such as 24 hours from the time the infusion started). Signs or symptoms of an IR include one or more of the following: paresthesia, chest pain, cough, nasal congestion, sneezing, throat irritation, pruritus, syncope, flushing, chills, fever, urticarial, angioedema, rash, skin reactions, itching, maculopapular rash, tachycardia, hypotension, dyspnea, nausea, vomiting, headache, back pain, chest discomfort or non-cardiac chest pain, abdominal pain, abdominal cramps, bronchospasm, laryngospasm, wheezing, respiratory tract congestion, excessive sweating, and erythema. (See, e.g., Doessegger et al. (2015) Clin & Trans Immunol. 4(7): e39 for further details.) Thus, in some embodiments, the individual does not experience any one or more of these signs or symptoms.

In some embodiments, the individual does not receive (e.g., require) premedication, i.e., medication administered prior the infusion of the anti-CD38 antibody (e.g. isatuximab) for the purpose of preventing or minimizing an IR. In some embodiments, the individual does not receive (e.g., require) medication (e.g., prophylactic medication) to prevent or minimize an IR following completion of the infusion of the anti-CD38 antibody (e.g., isatuximab). In some embodiments, the individual does not experience a delayed infusion reaction following administration (e.g., intravenous infusion) of the anti-CD38 antibody (such as isatuximab) at a dose of 10 mg/kg from a 250 ml volume. In some embodiments, the individual does not experience a delayed infusion reaction within any one of about 0.5, 1.0, 1.5, 2.0, 2.5, or 3.0 hours following administration (e.g., intravenous infusion) of the anti-CD38 antibody (such as isatuximab) at a dose of 10 mg/kg from a 250 ml volume. In some embodiments, the individual does not receive (e.g., require) post-medication, i.e., medication administered following the infusion of the anti-CD38 antibody (e.g. isatuximab) at a dose of 10 mg/kg from a 250 ml volume for the purpose of preventing or minimizing an IR. In some embodiments, the individual does not receive (e.g., require) post-medication within at least about any one of about 0.5, 1.0, 1.5, 2.0, 2.5 or 3 hours following the infusion of the anti-CD38 antibody (e.g. isatuximab) at a dose of 10 mg/kg from a 250 ml volume, e.g., for the purpose of preventing or minimizing an IR. In some embodiments the individual does not receive premedication or post-medication with any one or more of: an analgesic (e.g., acetaminophen or paracetamol), an H2 antagonist or antacid (such as ranitidine, cimetidine, omeprazole, or esomeprazole), an anti-inflammatory agent (such as a corticosteroid or a nonsteroidal anti-inflammatory drug), and/or an antihistamine (such as diphenhydramine, cetirizine, promethazine, dexchlorpheniramine) for the purpose of preventing or minimizing an IR prior to infusion of the anti-CD38 antibody (such as isatuximab) at a dose of 10 mg/kg from a 250 ml volume.

In some embodiments, the individual experiences a mild IR following administration of the anti-CD38 antibody (such as isatuximab). In some embodiments, the mild IR is no more than a Grade 1 or Grade 2 IR, as defined in the National Cancer Institute Common Terminology Criteria for Adverse Events, version 4.03 (NCI-CTCAE v. 4.03). The NCI-CTCAE v. 4.03 is publicly available online at evs(dot)nci(dot)nih(dot)gov/ftp1/CTCAE/About(dot)html. In some embodiments, the IR is a Grade 1 IR if the individual experiences a mild transient reaction (e.g., one or more of the signs/symptoms described herein, such as within 24 hours of the start of the infusion), wherein the interruption of the infusion is not indicated and/or wherein intervention is not indicated. In some embodiment, the IR is a Grade 2 IR if the individual experiences a reaction (e.g., one or more of the signs/symptoms described herein, such as within 24 hours of the start of the infusion), wherein infusion is interrupted and/or wherein intervention is indicated, and wherein the individual responds promptly to treatment (i.e., treatment of the one or more signs or symptoms of IR, such as those described herein), such as within about any one of 2, 4, 6, 8, 10, 12, 14, 16, 18, 20, or 14 hours of the treatment for the IR (including any range between these values). In some embodiments, the treatment for the IR comprises one or more of: short-term interruption of the infusion, administration of oxygen, administration of bronchodilators, administration of corticosteroids, administration of histamine blockers, and restarting the infusion at a slower rate

In some embodiments, the individual experiences a mild IR (e.g., a Grade 1 or Grade 2 IR) during or following the first intravenous infusion of 10 mg/kg of the anti-CD38 antibody (such as isatuximab) from a 250 ml fixed volume, e.g., during infusion on Day 1 of the first 28-day cycle. In some embodiments, the individual experiences no IR (or no further IR) during a second or subsequent infusion of the anti-CD38 antibody (e.g., isatuximab) from a 250 ml fixed volume. For example, in some embodiments, the individual experiences no IR (or no further IR) during infusion of 10 mg/kg of the anti-CD38 antibody (such as isatuximab) from a 250 ml fixed volume on any of Days 8, 15, and 22 of the first 28-day cycle and on any of Days 1 and 15 of any subsequent 28-day cycle.

In some embodiments, the individual does not experience a moderate or severe IR following infusion of an anti-CD38 antibody from a 250 ml volume, e.g., according to a method described herein. In some embodiments, the individual does not experience an IR of Grade 3, 4, or 5, as defined in the National Cancer Institute Common Terminology Criteria for Adverse Events, version 4.03 (NCI-CTCAE v. 4.03). In some embodiments, the IR is a Grade 3 IR if the individual experiences prolonged signs/symptoms of IR (such as described herein) and is not rapidly responsive to medication for the IR and/or to interruption of the infusion. In some embodiments, the IR is Grade 3 IR if the individual experiences recurrence of the signs/symptoms of IR (such as described herein) following initial improvement. In some embodiments, the IR is grade 3 IR is the individual requires hospitalization for the signs/symptoms of IR (such as described herein). In some embodiments, the IR is a Grade 4 IR if the signs/symptoms (such as described herein) are life threatening and/or require urgent intervention. In some embodiments, the IR is Grade 5 IR if the signs/symptoms of IR result in death.

In some embodiments, the dose of anti-CD38 antibody (such as isatuximab) administered from a 250 ml volume is not reduced during treatment, e.g., whether or not the individual experiences an IR.

### Articles of Manufacture or Kits

In another embodiment of the invention, an article of manufacture or a kit is provided comprising an anti-CD38 antibody (such as isatuximab). In some embodiments, the article of manufacture or kit further comprising carfilzomib, and/or dexamethasone. In some embodiments, the article of manufacture or kit further comprises package insert comprising instructions for using the anti-CD38 antibody (e.g., isatuximab) in conjunction with the carfilzomib and the dexamethasone to treat or delay progression of multiple myeloma (e.g., refractory multiple myeloma or relapsed and refractory multiple myeloma) in an individual who has received 1 to 3 prior therapies (or prior lines of therapy) for multiple myeloma. In some embodiments, the kit comprises isatuximab, carfilzomib, and dexamethasone.

The specification is considered to be sufficient to enable one skilled in the art to practice the invention. Various modifications of the invention in addition to those shown and described herein will become apparent to those skilled in the art from the foregoing description and fall within the scope of the appended claims. All publications, patents, and patent applications cited herein are hereby incorporated by reference in their entirety for all purposes.

### EXAMPLES

The present disclosure will be more fully understood by reference to the following examples. They should not, however, be construed as limiting the scope of the invention. It is understood that the examples and embodiments described herein are for illustrative purposes only and that various modifications or changes in light thereof will be suggested to persons skilled in the art and are to be included within the spirit and purview of this application and scope of the appended claims.

### Example 1A: A Phase III Randomized, Open-Label, Multicenter Study Comparing Isatuximab (SAR650984) in Combination with Carfilzomib and Low-Dose Dexamethasone Vs. Carfilzomib and Low-Dose Dexamethasone in Patients with Refractory or Relapsed and Refractory Multiple Myeloma

This Example describes a phase III, multicenter, multinational, randomized, open-label, parallel group, 2-arm study assessing the clinical benefit of isatuximab in combination with carfilzomib and dexamethasone (the “IKd” arm) versus carfilzomib and dexamethasone twice weekly (the “Kd” arm), in patients with relapsed and/or refractory multiple myeloma previously treated with 1 to 3 prior lines.

### I. Study Objectives

### A. Primary Objective

The primary objective (i.e., primary endpoint) of this study is to demonstrate the benefit of isatuximab in combination with carfilzomib and dexamethasone (IKd) in the prolongation of PFS using IMWG criteria as compared to carfilzomib and dexamethasone (Kd) in patients with relapsed and/or refractory MM previously treated with 1 to 3 lines of therapy.

Progression free survival is defined as the time from the date of randomization to the date of first documentation of progressive disease or the date of death from any cause, whichever comes first. Response and progression is determined according to IMWG criteria (see Kumar et al. (2016) “International Myeloma Working Group consensus criteria for response and minimal residual disease assessment in multiple myeloma.” Lancet Oncol. 17(8): e328-e346) and Durie et al. (2006) “International uniform response criteria for multiple myeloma. Leukemia. 20: 1467-1473). Progression based on paraprotein is confirmed based on 2 consecutive assessments.

The following disease assessment procedures are performed at screening (for eligibility), at Cycle 1 Day 1 prior to study treatment administration (baseline for response assessment), Day 1 of every cycle during treatment up to progression, end of treatment (EOT), and for patients who discontinue study treatment without PD, every month during follow-up until PD (even patients who would initiate further anti-myeloma therapy without PD):
M protein quantification (serum and 24-hour urine, protein immunoelectrophoresis, and immunofixation). After Cycle 1 Day 1, immunofixation is done in case of undetectable M protein (serum and urine).
Serum free light chain quantification.
Quantitative immunoglobulins.
Bone marrow aspiration (or biopsy as clinically indicated) at baseline (bone marrow disease involvement, FISH, and MRD), and then in case of VGPR or better.
Bone disease assessment:
   Skeletal survey or low-dose whole body computed tomography (CT) scan at baseline, then once a year, and anytime during the study if clinically indicated.
Extramedullary disease (plasmacytoma) assessment (including bone plasmacytoma):
   If known extramedullary disease at baseline, CT scan or magnetic resonance imaging (MRI) is done at baseline, repeated every 12 weeks (±1 week) until PD (even for patients who would initiate further anti-myeloma therapy without PD), and if clinically indicated.
   If suspected extramedullary disease (plasmacytoma) at baseline, CT scan or MRI is done at baseline and in case plasmacytoma is confirmed, CT scan or MRI is repeated every 12 weeks (±1 week) until PD (even for patients who would initiate further anti-myeloma therapy without PD), and if clinically indicated.
   At any time during study treatment in case of suspicion of progression of existing plasmacytoma or if clinically indicated in a patient with no previous positive image for extramedullary disease.

For bone lesion assessment and extramedullary disease, the same modality (skeletal survey or low-dose whole-body CT scan; CT scan or MRI) is be used throughout the study for each individual patient.

Progressive disease (IMWG criteria) is defined for patients with measurable serum and/or urine M protein as any one of the following (biological criteria in 2 consecutive assessments):
Increase of ≥25% in Serum M-component from nadir (the absolute increase must be ≥0.5 g/dL); serum M component increases ≥1 g/dL in 2 consecutive assessments are sufficient to define relapse if starting M component is ≥5 g/dL; and/or
Increase of ≥25% in Urine M-component from nadir (the absolute increase must be ≥200 mg/24 hour); and/or
Definite development of new bone lesions or soft tissue extramedullary disease or increase ≥50% from nadir in the sum of perpendicular diameters of existing soft tissue extramedullary disease lesions if >1 lesion or ≥50% increase in the longest diameter of a previous soft tissue extramedullary disease lesion >1 cm in short axis.

Clinical deterioration is considered progression in the primary analysis of PFS if an independent review committee considers reported clinical data as supporting clinical progression. In case of hypercalcemia, a full disease assessment is performed in order to identify any measurable parameter of myeloma progression (e.g., serum and urine M protein, lytic lesions assessment and plasmacytoma assessment) and potential alternative causes of hypercalcemia should be ruled out. Progression is not diagnosed on FLC progression only. Patients with only FLC measurable disease are not allowed in the protocol. If both serum and urine M protein becomes below level of eligibility on efficacy laboratory performed on Cycle 1 Day 1, progression and overall response are assessed according to the criteria in Tables A and B below.

TABLE A
Standard International Myeloma Working
Group (IMWG) Response Criteria

| Response | IMWG criteria |
|---|---|
| Complete | Negative immunofixation on the serum and urine and |
| Response | disappearance of any soft tissue plasmacytomas and |
| (CR) | <5% plasma cells in BMAs. |
| | Two consecutive assessments are needed. |
| | No known evidence of progressive disease or |
| | new bone lesions if radiographic studies |
| | were performed |
| Stringent | CR as defined above plus: |
| Complete | Normal FLC ratio (0.26 to 1.65) and |
| Response | Absence of clonal cells in BM by immunohistochemistry |
| (sCR) | (κ/λ ratio ≤4:1 or ≥1:2 for κ and λ patients, |
| | respectively, after counting ≥100 plasma cells). |
| | Two consecutive assessments of laboratory parameters |
| | are needed. No known evidence of progressive disease |
| | or new bone lesions if radiographic studies |
| | were performed |
| Very Good | Serum and urine M protein detectable by |
| Partial | immunofixation but not on electrophoresis or |
| Response | ≥90% reduction in serum M protein |
| (VGPR) | plus urine M protein level <100 mg/24 hour. |
| | ≥90% decrease in the sum of the products |
| | of maximal perpendicular diameter compared |
| | to baseline in soft tissue plasmacytoma. |
| | Two consecutive assessments are needed. |
| | No known evidence of progressive disease |
| | or new bone lesions if radiographic studies |
| | were performed. |
| Partial | ≥50% reduction of serum M protein and |
| Response | reduction in 24 hours urinary M protein |
| (PR) | by ≥90% or to <200 mg/24 hour |
| | In addition to the above listed criteria, if |
| | present at baseline, a ≥50% reduction in the size |
| | (sum of the products of the maximal perpendicular |
| | diameters) of soft tissue plasmacytomas is |
| | also required. |
| | Two consecutive assessments are |
| | needed. No known evidence of progressive disease |
| | or new bone lesions if radiographic studies |
| | were performed. |
| Minimal | ≥25% but ≤49% reduction in serum M protein |
| Response | and reduction in 24 h urine M protein by 50 |
| (MR) | to 89%, which still exceed 200 mg/24 hour. |
| | In addition to the above listed criteria, |
| | if present at baseline, ≥50% reduction |
| | in size (SPD) of soft tissue plasmacytomas |
| | is also required. |
| | Two consecutive assessments are needed. No known |
| | evidence of progressive disease or new bone lesions |
| | if radiographic studies were performed. |
| Stable | Not meeting criteria for CR, VGPR, PR, MR or |
| Disease | progressive disease. |
| (SD) | No known evidence of progressive disease |
| | or new bone lesions if |
| | radiographic studies were performed. |
| Progressive | Any 1 or more of the following criteria: |
| Disease | Increase of ≥25% from lowest confirmed |
| (PD) | value in any 1 of the following criteria: |
| | Serum M protein (the absolute increase must |
| | be ≥0.5 g/dL). |
| | Serum M protein increase ≥1 g/dL |
| | if the lowest M component was ≥5 g/dL. |
| | Urine M-component (the absolute |
| | increase must be ≥200 mg/24 hour). |
| | Appearance of new lesion(s), ≥50% increase |
| | from nadir in SPD of >1 lesion, or ≥50% |
| | increase in the longest diameter of a |
| | previous lesion >1 cm in short axis. Two |
| | consecutive assessments are needed for PD |
| | on M protein. |

TABLE B
IMWG Minimal Residual Disease Criteria
(requires CR as defined in Table A)

| Response | IMWG criteria |
|---|---|
| Sustained | MRD negativity in the marrow (NGF or NGS, |
| MRD-negative | or both) and by imaging as defined below, |
| | confirmed minimum of 1 year apart. |
| | Subsequent evaluations can be used to |
| | further specify the duration of negativity |
| | (e.g., MRD-negative at 5 years) |
| Flow MRD- | Absence of phenotypically aberrant clonal |
| negative | plasma cells by NGF on BMAs using the |
| | EuroFlow standard operation procedure for |
| | MRD detection in multiple myeloma (or |
| | validated equivalent method) with a |
| | minimum sensitivity of 1 in 10⁵ |
| | nucleated cells or higher |
| Sequencing | Absence of clonal plasma cells by NGS on BMA |
| MRD-negative | in which presence of a clone is defined as |
| | less than two identical sequencing reads |
| | obtained after DNA sequencing of BMAs using |
| | the LymphoSIGHT platform (or validated |
| | equivalent method) with a minimum sensitivity |
| | of 1 in 10⁵ nucleated cells or higher |
| Imaging- | MRD negativity as defined by NGF or NGS plus |
| positive | disappearance of every area of increased |
| MRD-negative | tracer uptake found at baseline or a preceding |
| | PET/CT or decrease to less mediastinal blood pool |
| | SUV or decrease to less than that of surrounding |
| | normal tissue |

Abbreviations for Tables A and B: CR = complete response, FLC = free light chain, IMWG = International Myeloma Working Group, M = monoclonal, MRD = minimal residual disease, NGF = next-generation flow, NGS = next-generation sequencing, PD = progressive disease, PET = positron emission tomography, MR = minor response, PR = partial response, sCR = stringent complete response, SD = stable disease, SPD = sum of the products of the maximal perpendicular diameters of measured lesions, SUV = maximum standardized uptake value, VGPR = very good partial response.

### B. Key Secondary Efficacy Endpoints

The key secondary efficacy endpoints are:
ORR: Best overall response per patient is assessed in order to determine ORR, defined as the proportion of patients with stringent complete response (sCR), CR, VGPR, and PR as best overall response as assessed using the IMWG response criteria (See Table A). Bone marrow biopsy is done for sCR assessment as per investigator decision.
Rate of VGPR or better: Defined as the proportion of patients with sCR, CR, and VGPR.
Rate of VGPR or better with MRD negativity: Defined as the proportion of patients for whom MRD assessed by sequencing is negative at any time after first dose of study treatment. Minimal residual disease is assessed by next-generation sequencing in bone marrow (BM) samples from patients who achieve VGPR or better, to determine the depth of response at the molecular level. Threshold for negativity is at least 10⁻⁵. Bone marrow aspirates (BMA) are collected at screening and at the time of VGPR or better confirmation. If the patient presents with VGPR or better but is determined MRD positive, another BM sample is collected 3 months (3 cycles) later, in order to identify late negativity. A third sample is collected after another 3 months, if the patient remains MRD positive and is still being treated. No more than 3 on-treatment bone marrow samples are obtained unless a patient achieves CR after a third BM sample MRD positive performed during VGPR. In this case no more than 3 additional BM samples are collected. Therefore, a maximum of 6 BMA are performed by patient (no more than 3 per category of response). However, because BMA is invasive procedure, the following guidance is given in the purpose to limit as much as possible the number of BMA.
   For patients with CR without previous documentation of VGPR: First bone marrow for MRD assessment is collected at time of confirmation of CR (i.e., at the second time point showing CR). If patient is determined MRD positive, another BM sample is collected 3 months (3 cycles) later, in order to identify late negativity. A third sample is collected after another 3 months if the patient remains MRD positive and is still being treated.
   For patients with VGPR: First bone marrow is collected when VGPR is confirmed at the second time point or at a later time point as per investigator judgement based on kinetic of M protein decrease and/or if a plateau phase is reached (plateau is defined as variation less than 20% over 12 weeks). If MRD is positive on first BMA, a second BMA is collected 3 months later (3 cycles) to identify late negativity. In case of MRD is still positive on second BMA made while patient is in VGPR, the timing to perform the third BMA can be postponed until CR achievement. In case of the patient becomes CR and patient was MRD positive on the last BMA performed during VGPR, a BMA will be done for MRD assessment at time of confirmation of CR. After the first BMA during CR is done and in case patient was MRD positive on this BMA, the additional BMA planned by the protocol can be discussed with the patient.
CR rate: Defined as the proportion of patients with sCR and CR. Patients with demonstrated isatuximab interference will be considered in the BOR category corresponding to the M protein assessment obtained without interference, when the antibody-capture interference assay will be available.
OS: Defined as the time from the date of randomization to death from any cause.

### C. Other Secondary Efficacy Endpoints

Other secondary efficacy endpoints are evaluated as follows:
Duration of Response (DOR): Defined as the time from the date of the first IRC determined response for patients achieving PR or better to the date of first documented progressive disease (PD) or death, whichever happens first.
Time to Progression (TTP): Defined as time from randomization to the date of first documentation of PD.
PFS2: Defined as time from the date of randomization to the date of first documentation of PD after initiation of further anti-myeloma treatment or death from any cause, whichever happens first.
Time to first response: Defined as the time from randomization to the date of first response (PR or better) that is subsequently confirmed.
Time to best response: Defined as the time from randomization to the date of first occurrence of best overall response (PR or better) that is subsequently confirmed.

### D. Safety Endpoints

Safety in terms of treatment emergent adverse events (TEAEs), adverse events (AEs), serious adverse events (SAEs), infusion associated reactions (IARs), Eastern Cooperative Oncology Group Performance Status (ECOG PS, see Oken et al. Toxicity and Response Criteria of the Eastern Cooperative Oncology Group. Am J Clin Oncol. 1982; 5:649-55), laboratory parameters, vital signs, and findings from physical examination are assessed through the study and are reported.

Adverse event data are collected throughout the study. Treatment-emergent AEs are defined as AEs that develop, worsen, or become serious during the treatment period. The treatment period is defined as the time from first dose of study treatment up to 30 days after last dose of study treatment. Adverse events and laboratory parameters will be graded using NCI-CTCAE v4.03 (see, e.g., https://www(dot)eortc(dot)be/services/doc/ctc/CTCAE_4.03_2010-06-14_QuickReference_5×7.pdf).

### E. Patient-Reported Outcomes

Patient-reported outcome measures include the European Organisation for Research and Treatment of Cancer (EORTC) Quality of Life questionnaire with 30 questions (QLQ C30), the EORTC myeloma module with 20 items (QLQ-MY20), and the European Quality of Life Group measure with 5 dimensions and 5 levels per dimension (EQ-5D-5L). (See, e.g., https://qol(dot)eortc(dot)org/questionnaires/ and https://euroqol(dot)org/eq-5d-instruments/eq-5d-5l-about/).

All 3 questionnaires are designed for self-completion. All patient-reported outcomes are completed at the site by the patient. To minimize any bias, patients fill out the ePROs before clinician assessments and discussion of their clinical condition, treatment plan, AEs, and any other related topics that could influence patient's perception and feelings prior to responding to the questions.

### F. Pharmacokinetics

Pharmacokinetic (PK) evaluation for isatuximab is performed in all patients in the IKd arm. Blood samples are collected from all patients treated with isatuximab up to Cycle 10 using a sparse sampling strategy in order to assess the PK profile of isatuximab using population PK approach. In a subset of approximately 12 patients from the IKd arm, blood samples are collected at selected time points at Cycle 1 Day 15 for carfilzomib PK evaluation. The PK parameters that are measured include, but are not limited to, those listed in Table C below.

TABLE C
Exemplary Pharmacokinetic Parameters

| Parameters | Definition |
|---|---|
| Cₑₒᵢ | Concentration observed at the end of IV infusion |
| Cₘₐₓ | Maximum concentration observed after the first infusion |
| tₘₐₓ | Time to reach Cₘₐₓ |
| Cₗₐₛₜ | Last concentration observed above the lower limit of |
| | quantification |
| tₗₐₛₜ | Time of Cₗₐₛₜ |
| C_{trough} | Plasma concentration observed just before treatment |
| | administration during repeated dosing |
| AUCₗₐₛₜ | Area under the plasma concentration versus time curve |
| | calculated using the trapezoidal method from time 0 to tₗₐₛₜ |
| AUC | Area under the plasma concentration versus time curve |
| | extrapolated to infinity according to the following equation: |
| | AUC = AUCₗₐₛₜ + C_{last/λz} |

### F. Immunogenicity

Human anti-drug antibodies (ADAs) to isatuximab will be assessed for the IKd patients only on Day 1 prior to isatuximab administration from Cycle 1 to Cycle 10.

### G. Exploratory Endpoints

A blood sample is collected on Day 1 of Cycle 1. Leukocyte DNA is extracted and analyzed for immune genetic determinants (such as Fcγ receptor polymorphisms) and correlated with parameters of clinical response.

In the IKd arm only, an additional blood sample is collected at all time-points to evaluate the potential interference of isatuximab in the M protein assessment up to Cycle 30. This sample is collected after Cycle 30 and until disease progression only for patients who reach at least VGPR at this cycle. In case of isatuximab is stopped before progression, sample interference assay is collected up to 3 months or PD, whichever comes first. After Cycle 1 Day 1, immunofixed sample is analyzed in case of M protein is 0 g/dL in all patients. In addition, in order to identify patients with potential isatuximab interference, immunofixed samples are analyzed also in patients with serum M protein ≤0.2 g/dL.

In addition to the 3 cytogenetic abnormalities (del(17p), t(4:14) and t(14:16)) assessed by fluorescence in-situ hybridization (FISH) at baseline to determine R-ISS stage which is a stratification factor, other cytogenetic abnormalities such as but not limited to del (1p) and gain (1q) deletion are assessed and correlated with parameters of clinical response.

Each of the assessments described above are chosen for use in this study and are considered well established and relevant in a hemato-oncology setting.

### II. Study Design

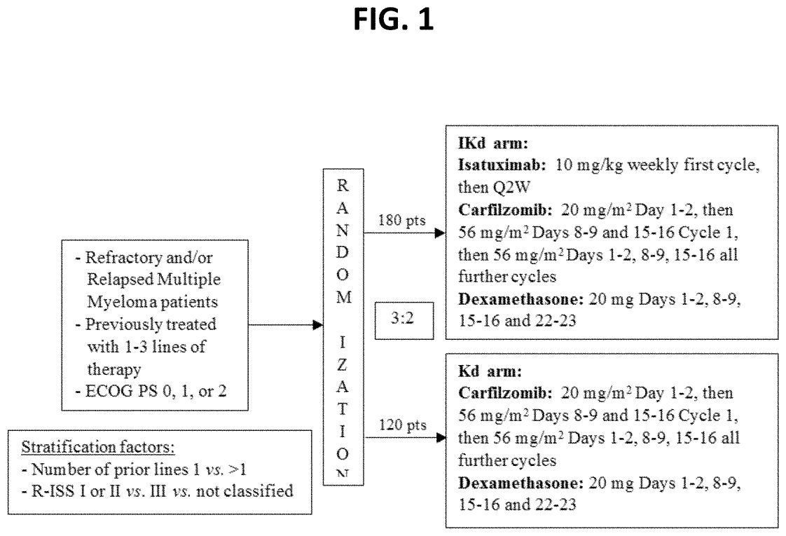

After confirmation of eligibility criteria (which are described in further detail below), patients are randomly assigned using an interactive response technology (IRT) system in a 3:2 ratio (experimental arm:control arm) to one of the two arms shown in Table D below. The total expected number of patients is 300 (180 patients in the IKd arm, and 120 patients in the Kd arm). A schematic of the study design is also provided in FIG. 1.

TABLE D
Study Treatment Arms

| TREATMENT | Cycle 1 | Cycles ≥2 |
|---|---|---|
| ARM | (28 day cycle) | (28 day cycles) |
| IKd | Isatuximab: 10 mg/kg | Isatuximab: 10 mg/kg |
| (experimental) | on Days 1, 8, 15, and 22 | on Days 1 and 15 |
| isatuximab + | Carfilzomib: 20 mg/m² | Carfilzomib: 56 mg/m² |
| carfilzomib + | on Days 1-2, then 56 mg/m² | on Days 1-2, 8-9 and |
| dexamethasone | on Days 8-9 and 15-16 | 15-16 |
| | Dexamethasone: 20 mg | Dexamethasone: 20 mg |
| | on Days 1-2, 8-9, 15-16, | on Days 1-2, 8-9, 15-16, |
| | and 22-23 | and 22-23 |
| Kd | Carfilzomib: 20 mg/m² | Carfilzomib: 56 mg/m² |
| (control) | on Days 1-2, then 56 mg/m² | on Days 1-2, 8-9 and |
| carfilzomib + | on Days 8-9 and 15-16 | 15-16 |
| dexamethasone | Dexamethasone: 20 mg | Dexamethasone: 40 mg |
| | on Days 1-2, 8-9, 15-16, | on Days 1, 8, 15, and |
| | and 22-23 | 22 |

Randomization was stratified by number of previous lines of therapy (1 vs. more than 1) and R-ISS stage (I or II vs. III vs. not classified). See Palumbo A, et al. Revised International Staging System for Multiple Myeloma: A Report From International Myeloma Working Group. J Clin Oncol. 2015; 33(26):3863-9. Patients are treated until disease progression, unacceptable adverse events (e.g., unacceptable toxicity), or patient wish, whichever comes first.

### A. Duration of Study Participation for Each Patient

Each patient is considered in the study from informed consent signature until death, consent withdrawal or overall survival analysis cut-off date, whichever comes first.

The duration of the study for a patient includes a period for screening of up to 3 weeks. The duration of each treatment cycle was 28 days. Patients continue study treatment until disease progression, unacceptable AEs, patient wish, or any other reason. All AEs occurring after informed consent signature are reported up to 30 days after last study treatment administration.

After study treatment discontinuation, patients return to the study site 30 days after the last dose of study treatment for end of treatment or before further anti myeloma therapy initiation, whichever comes first, end of treatment (EOT) assessments, and 90 days after the last dose of study treatment for HRQL questionnaires.

Related AEs and all SAEs regardless of relationship to the study treatment ongoing at the time of study treatment discontinuation are followed during the follow-up period until resolution or stabilization. During the follow-up period, all (serious or non-serious) new AEs related to study treatment and any second primary malignancy are collected and followed up until resolution or stabilization.

Patients who discontinue the study treatment due to progressive disease (PD) are followed up every 3 months (12 weeks) for HRQL at first FU visit (90 days after the last study treatment), further anti-myeloma therapies, second primary malignancies, PFS2, and survival until death, or the final PFS analysis cut-off date, whichever comes first. Patients who discontinue the study treatment prior to documentation of PD are followed-up every 4 weeks until disease progression (even for patients who would initiate further anti-myeloma therapy without PD), HRQL 90 days after last study treatment, and then after confirmation of disease progression, every 3 months (12 weeks) for further anti-myeloma therapies, second primary malignancies, PFS2, and survival, until death or the final PFS analysis cut-off date, whichever comes first.

For all alive patients at the final PFS analysis cut-off date, survival status is collected approximately one year after the final PFS analysis cut-off date and thereafter on a yearly basis up to 3 years after the final PFS analysis cut-off date.

Patients still on treatment at the time of the final PFS analysis cut-off date or OS analysis cut-off date, and benefitting from the study treatment, continue the study treatment until disease progression, unacceptable AEs, patient wish to discontinue further study treatment, or any other reasons. For cycles administered after the cut-off dates, all ongoing SAEs (related or not) and all ongoing related non serious AEs at this cut-off date, all new related AEs (serious or not), IP administration, and reason of EOT continue to be collected. If a patient received less than 10 cycles at the final PFS analysis cut-off date, anti-drug antibodies (ADA)/PK samples is stopped. In case of the last ADA is positive or inconclusive, an additional ADA is sampled 3 months later. No further ADA is sampled, even if this 3-month sample is positive.

### B. Determination of End of Clinical Trial (all Patients)

The PFS analysis (primary endpoint analysis) is event driven and the final PFS analysis cut-off date is the date when 159 PFS events (progression or death, whichever comes first) have occurred (around 36 months from first patient being randomized). The OS analysis cutoff date is approximately 3 years after the primary PFS analysis cut-off date. The primary analysis of PFS corresponds either to the positive interim analysis or the final PFS analysis.

### III. Selection of Patients

### A. Inclusion Criteria

Eligible patients are considered for inclusion if they meet all of the following criteria:
Multiple myeloma.
Measurable disease: Serum M protein ≥0.5 g/dL measured using serum protein immunoelectrophoresis and/or urine M protein ≥200 mg/24 hours measured using urine protein immunoelectrophoresis.
Patient with relapsed and/or refractory MM with at least 1 prior line and no more than 3 prior lines, including IMiDs® and proteasome inhibitors.
Patient has given voluntary written informed consent before performance of any study related procedures not part of normal medical care.

A line of therapy consists of ≥1 complete cycle of a single agent, a regimen consisting of a combination of several drugs, or a planned sequential therapy of various regimens. A treatment is considered a new line of therapy if any 1 of the following 3 conditions are met (see, e.g., Rajkumar et al. Guidelines for the determination of the number of prior lines of therapy in multiple myeloma. Blood 2015; 127(7):921-2).

i. Start of a New Line of Treatment after Discontinuation of a Previous Line.
If a treatment regimen is discontinued for any reason and a different regimen is started, it should be considered a new line of therapy. A regimen is considered to have been discontinued if all the drugs in that given regimen have been stopped. A regimen is not considered to have been discontinued if some of the drugs of the regimen, but not all, have been discontinued. The reasons for discontinuation, addition, substitution, or stem cell transplantation (SCT) do not influence how lines are counted. It is recognized that reasons for change may include end of planned therapy, toxicity, progression, lack of response, inadequate response.

ii. The Unplanned Addition or Substitution of 1 or More Drugs in an Existing Regimen.
Unplanned addition of a new drug or switching to a different drug (or combination of drugs) due to any reason is considered a new line of therapy.
Stem Cell Transplantation (SCT): In patients undergoing >1 SCT, except in the case of a planned tandem SCT with a predefined interval (such as 3 months), each SCT (autologous or allogeneic) should be considered a new line of therapy regardless of whether the conditioning regimen used is the same or different. It is recommended that data on type of SCT also be captured. Planned tandem SCT is considered 1 line. Planned induction and/or consolidation, maintenance with any SCT (frontline, relapse, autologous, or allogeneic) is considered 1 line.

iii. Interruptions and Dose Modifications
If a regimen is interrupted or discontinued for any reason and the same drug or combination is restarted without any other intervening regimen, then it should be counted as a single line. However, if a regimen is interrupted or discontinued for any reason, and then restarted at a later time point but 1 or more other regimens were administered in between, or the regimen is modified through the addition of 1 or more agents, then it should be counted as 2 lines. Modification of the dosing of the same regimen should not be considered a new line of therapy.

### B. Exclusion Criteria

Patients who meet all the inclusion criteria above are screened for the following exclusion criteria:
Less than 18 years (or country's legal age of majority if the legal age is >18 years).
Primary refractory MM, defined as patients who have never achieved at least a MR with any treatment during the disease course.
Patient with serum free light chain (FLC) measurable disease only.
Patient with prior anti-CD38 mAb treatment with progression on or within 60 days after end of anti-CD38 mAb treatment or failure to achieve at least MR to treatment (i.e., refractory to anti-CD38).
Any anti-myeloma drug treatment within 14 days before randomization, including dexamethasone.
Patient who has received any other investigational drugs or prohibited therapy for this study within 28 days prior to randomization.
Prior treatment with carfilzomib.
Known history of allergy to CAPTISOL® (a cyclodextrin derivative used to solubilize carfilzomib), prior hypersensitivity to sucrose, histidine (as base and hydrochloride salt), polysorbate 80, or any of the components (active substance or excipient) of study treatment that are not amenable to premedication with steroids, or H2 blockers, that would prohibit further treatment with these agents.
Patients with contraindication to dexamethasone.
Prior allogenic hematopoietic stem cell transplant with active graft versus host disease (any grade and/or being under immunosuppressive treatment within 2 months before randomization).
Known amyloidosis or concomitant plasma cell leukemia.
Pleural effusions requiring thoracentesis or ascites requiring paracentesis or any major procedures within 14 days before randomization: e.g., plasmapheresis, curative radiotherapy, major surgery (kyphoplasty is not considered a major procedure).
Eastern Cooperative Oncology Group (ECOG) performance status (PS) >2.
Platelets <50,000 cells/4 if <50% of BM nucleated cells are plasma cells and <30,000 cells/4 if ≥50% of BM nucleated cells are plasma cells. Platelet transfusion is not allowed within 3 days before the screening hematological test.
Absolute neutrophil count (ANC)<1000 μ/L (1×10⁹/L). The use of granulocyte colony stimulating factor (G-CSF) is not allowed to reach this level.
Creatinine clearance <15 mL/min/1.73 m² (Modification of Diet in Renal Disease [MDRD] Formula: Glomerular filtration rate (mL/min/1.73 m²)=175×(Scr)−1.154×(Age)−0.203×(0.742 if Female)×(1.212 if African American); Scr is serum creatinine expressed in mg/dL; age is expressed in years).
Total bilirubin >1.5× upper limit of normal (ULN), except for known Gilbert syndrome.
Corrected serum calcium >14 mg/dL (>3.5 mmol/L).
Aspartate aminotransferase (AST) and/or alanine aminotransferase (ALT]) >3×ULN.
Ongoing toxicity (excluding alopecia and those listed in eligibility criteria) from any prior anti-myeloma therapy of Grade >1 (National Cancer Institute Common Terminology for Adverse Events [NCI-CTCAE] v4.03)
Prior malignancy. Adequately treated basal cell or squamous cell skin or superficial (pTis, pTa, and pT1) bladder cancer or low risk prostate cancer or any in situ malignancy after curative therapy are allowed, as well as any other cancer for which therapy has been completed ≥5 years prior to randomization and from which the patient has been disease-free for ≥5 years.
Any of the following within 6 months prior to randomization: myocardial infarction, severe/unstable angina pectoris, coronary/peripheral artery bypass graft, New York Heart Association class III or IV congestive heart failure (CHF), Grade ≥3 arrhythmias, stroke, or transient ischemic attack.
Left ventricular ejection fraction (LVEF)<40%.
Known acquired AIDS related illness or HIV disease requiring antiretroviral treatment, or to have active hepatitis A, B (defined as a known positive hepatitis B surface antigen (HBsAg) result), or C (defined as known quantitative HCV RNA results greater than the lower limits of detection of the assay or positive HCV antigen) infection.
Any of the following within 3 months prior to randomization: treatment resistant peptic ulcer disease, erosive esophagitis or gastritis, infectious or inflammatory bowel disease, diverticulitis, pulmonary embolism, or other uncontrolled thromboembolic event.
Any severe acute or chronic medical condition which could impair the ability of the patient to participate in the study or interfere with interpretation of the study results (e.g., systemic infection unless anti-infective therapy is employed), or patient unable to comply with the study procedures.
Female patients who are pregnant or lactating.
Women of childbearing potential (WOCBP) not protected by highly-effective method of birth control and/or who are unwilling or unable to be tested for pregnancy.
Male participant with a female partner of childbearing potential not protected by highly-effective method of birth control.

### IV. Study Treatments

### A. Investigational Medicinal Products (IMPs)

i. Isatuximab (IV Administration)

Isatuximab is formulated as a concentrated solution for infusion in vials containing 20 mg/mL (500 mg/25 mL) isatuximab in 20 mM histidine, 10% (w/v) sucrose, 0.02% (w/v) polysorbate 80, pH 6.0 buffer. Isatuximab is supplied for parenteral administration as a sterile, nonpyrogenic, injectable, colorless, 20 mg/mL concentrate for solution for infusion that may contain white to off-white particulates and was packaged in 30 mL glass vials fitted with elastomeric closure. Each vial contains a nominal content of 500 mg of isatuximab. The fill volume is established to ensure removal of 25 mL. For administration to patients, the appropriate volume of isatuximab is diluted in an infusion bag of 0.9% sodium chloride solution. The final infusion volume corresponding to the dose of isatuximab is administered for a period of time that depends on dose administered and was based on protein amount given per hour.

Isatuximab is administered at a dose of 10 mg/kg to patients in the IKd arm via intravenous infusion on Days 1, 8, 15, and 22 for the first 28-day cycle, and then on Days 1 and 15 for each subsequent 28-day cycle. (All cycles were 28 days in duration.) Dose modifications (described in further detail below) were applied in cases of toxicity.

ii. Carfilzomib (IV Administration)

Carfilzomib (Kyprolis®) from available commercial supplies is used for this study where applicable; otherwise, it is re-labeled by the sponsor according to Good Manufacturing Practice (GMP) guidelines before supplies are provided to the study sites. Details regarding the formulation, storage, and handling procedures for carfilzomib are provided in the commercial package insert. The lyophilized product is reconstituted with water for injection to a final carfilzomib concentration of 2 mg/mL prior to administration.

iii. Dexamethasone (Oral or IV Administration)

Dexamethasone from available commercial supplies is used for this study where applicable; otherwise, it is re-labeled by the sponsor according to Good Manufacturing Practice (GMP) guidelines before supplies are provided to the study sites. Details regarding the formulation, and handling procedures for dexamethasone are provided in the commercial package insert.

### B. Non-Investigational Medicinal Products (NIMPs)—Premedication for the Prevention of Infusion Reactions (IRs)

All patients allocated to IKd arm receive premedication prior to isatuximab infusion in order to reduce the risk and severity of IARs commonly observed with administration of monoclonal antibodies. The recommended premedication agents are: diphenhydramine 25-50 mg IV (or equivalent: e.g., cetirizine, promethazine, dexchlorpheniramine, according to local approval and availability. Intravenous route was preferred for at least the first 4 infusions), dexamethasone per os/IV (dose provided below), ranitidine 50 mg IV (or equivalent: other approved H2 antagonists (e.g., cimetidine), oral proton pump inhibitors (e.g., omeprazole, esomeprazole) and acetaminophen 650-1000 mg per os 15 to 30 minutes (but no longer than 60 minutes) prior to isatuximab infusion. Once the premedication regimen is completed, the isatuximab infusion starts immediately.

On the day of isatuximab infusion, the following NIMPs are administered in the following order:
Acetaminophen (paracetamol) 650 mg to 1000 mg per os; then
Ranitidine 50 mg IV (or equivalent); then
Diphenhydramine 25 mg to 50 mg IV (or equivalent); then
Dexamethasone 20 mg IV (which is also part of study treatment).

When dexamethasone was administered intravenously, the premedications were administered in the following order:
Acetaminophen 650 mg to 1000 mg per os; then
Ranitidine 50 mg IV (or equivalent); then
Diphenhydramine 25 mg to 50 mg IV (or equivalent); then
Dexamethasone 40 mg IV (or 20 mg IV for patients ≥75 years of age).

In regions where there is no IV formulation of diphenhydramine or equivalent, per os formulation is allowed from the first isatuximab infusion. In this case, it is taken one to two hours prior to isatuximab infusion start.

When carfilzomib is administered without isatuximab (patients allocated to the Kd arm and on Days 2, 8, and 16 for patients allocated to the IKd arm), dexamethasone is administered at least 30 minutes prior to carfilzomib infusion.

In case of dexamethasone being prematurely stopped and other study treatment being continued, steroid premedication can be considered with methylprednisolone 100 mg IV if IAR premedication is still needed for isatuximab and/or carfilzomib according to investigator judgment.

For the patients who do not experience an IAR upon 4 consecutive administrations of isatuximab, the Investigator may reconsider the need of specific isatuximab premedication for IAR.

### V. Dosage and Schedule

There is no limitation in the number of cycles that are administered in the absence of major toxicity, disease progression, or any other discontinuation criteria. PD diagnosis made on laboratory criteria is confirmed by 2 consecutive measures before to treatment discontinuation. The treatment is continued until confirmation of the PD.

Dose adjustment (dose delay, dose omission, and for carfilzomib and dexamethasone dose reduction) is permitted for subsequent treatment cycles based on individual patient tolerance. Additional details regarding dose adjustments are provided below. No dose reductions are allowed for isatuximab infusion.

A. Study treatments (IMP)

Study treatment is defined as isatuximab/carfilzomib/dexamethasone in IKd experimental arm and carfilzomib/dexamethasone in Kd control arm.

Both isatuximab and carfilzomib can induce IARs, and premedication is required prior to their administration.

Patients allocated to IKd arm routinely receive premedications, which also include dexamethasone, prior to isatuximab infusion to reduce the risk and severity of IARs commonly observed with mAbs and with carfilzomib. Dexamethasone is administered prior to carfilzomib for patients allocated to the Kd arm. For patients allocated to IKd arm, dexamethasone is administered prior to carfilzomib when there is no isatuximab infusion (such as on Days 2, 9, and 16 at Cycle 1 and on Days 2, 8, 9, and 16 at further cycles).

Hydration is required prior to the 2 first carfilzomib administrations (on Day 1 and Day 2 Cycle 1). Hydration should be started orally at least 48 h prior Day 1 Cycle 1. Hydration for further infusions within cycle 1 and further cycles is left to Investigator judgment. (Details for hydration are given below.) Patients with a body surface area (BSA) >2.2 m² will use 2.2 m² for the determination of carfilzomib dose.

i. IKd Arm (Experimental Arm)

Drug administration (after pre-medication as described below) for patients treated with isatuximab, carfilzomib, and dexamethasone combination is as follows:

Dexamethasone 20 mg on Days 1, 2, 8, 9, 15, 16, 22, and 23 in a 28-day cycle, between 15 to 30 minutes (but no longer than 60 minutes) prior to isatuximab or at least 30 minutes prior to carfilzomib on the days when there is no isatuximab administration. Dexamethasone is administered IV on the days of isatuximab and/or carfilzomib administration and PO on the other days. No post infusion prophylaxis with dexamethasone is required.

Isatuximab is administered IV at a dose of 10 mg/kg weekly for the first month (e.g., 28-day cycle), then Q2W for each 28-day cycle thereafter. The rate of infusion for isatuximab is initiated at 175 mg/hour. First infusion: Infusion is initiated at 175 mg/hour. In the absence of IARs after 1 hour of infusion, infusion rate is increased by 50 mg/hour increments every 30 minutes, to a maximum of 400 mg/hour. Subsequent infusions: Infusion is initiated at 175 mg/hour. In the absence of IAR after 1 hour of infusion, infusion rate is increased by 100 mg/hour increments every 30 minutes, to a maximum of 400 mg/hour.

Carfilzomib (after appropriate hydration) is administered IV over 30 minutes at a dose of 20 mg/m² on Days 1 and 2, 56 mg/m² on Days 8, 9, 15, and 16 of Cycle 1, and then 56 mg/m² on Days 1, 2, 8, 9, 15, and 16 of all further cycles. The carfilzomib infusion follows the isatuximab infusion and begins just after the end of the isatuximab infusion. The dose is escalated to 56 mg/m² on Day 8 and for further administrations if the patient does not experience any toxicity Grade >2 (except non-complicated hematological toxicity (toxicity meaning related to study treatment) or recovered tumor lysis syndrome (TLS)).

ii. Kd Arm (Control Arm)

Drug administration for patients treated with carfilzomib and dexamethasone combination is performed as follows:

Dexamethasone 20 mg on Days 1, 2, 8, 9, 15, 16, 22, and 23, at least 30 mins prior to carfilzomib on the days of carfilzomib administration. Dexamethasone is administered IV on the days of carfilzomib administration and PO on the other days.

Carfilzomib (after appropriate hydration) is administered IV over 30 mins at a dose of 20 mg/m² on Days 1 and 2, 56 mg/m² on Days 8, 9, 15, and 16 of Cycle 1, then 56 mg/m² on Days 1, 2, 8, 9, 15, and 16 of all further cycles. The dose will be escalated to 56 mg/m² on Day 8 and for further administrations if the patient does not experience any toxicity higher than Grade 2 (except non-complicated hematological toxicity (toxicity meaning related to study treatment) or recovered TLS).

### B. Carfilzomib Hydration

At least 48 hours before Cycle 1 Day 1, oral hydration is given as follows: 30 mL/kg/day (approximately 6 to 8 cups of liquid per day) continuing up to the time of treatment. Patient compliance is assessed before initiating treatment, which is to be delayed if oral hydration is not adequate. Oral hydration is continued for infusions within Cycle 1 and in Cycle 2 and beyond at the Investigator's discretion. In case tumor lysis syndrome (TLS) occurs after prior study treatment administration, hydration for subsequent infusions is done as needed as per investigator judgement.

Patients with a history of cardiac disease (such as CHF and cardiomyopathy) or pulmonary edema are monitored closely for signs of fluid overload. Patients with hypertension in medical history have controlled blood pressure before treatment initiation.

Intravenous hydration is given immediately prior to carfilzomib on D1 and D2 during Cycle 1, and at the Investigator's discretion after Cycle 1. Intravenous hydration consists of 500 mL normal saline or other appropriate IV fluid prior to carfilzomib infusion over 30 to 60 minutes. The goal of the hydration program is to maintain robust urine output (e.g., ≥2 L/day). Patients are monitored periodically during this period for evidence of fluid overload.

On the days where both isatuximab and carfilzomib are administered, the volume of isatuximab infusion is considered in hydration required prior to carfilzomib infusion. If the volume of isatuximab infusion does not reach at least 500 mL, additional hydration is administered to reach at least 500 mL. In this case, additional volume is administered prior start of isatuximab infusion. Total volume of hydration can be less than 500 mL (no less than 250 mL) or kept at 500 mL. Hydration is administered over a longer time for patients with borderline left ventricular ejection fraction (LVEF) and/or for whom there is a risk of cardiac decompensation according to investigator's judgement. The carfilzomib infusion is started after isatuximab infusion is completed.

### C. Dose Modifications

Dose adjustment (dose delay, dose omission, and dose reduction (for carfilzomib and/or dexamethasone only)) is permitted for subsequent treatment cycles based on individual patient tolerance. Patients may have a dose omitted (isatuximab and/or carfilzomib and/or dexamethasone) within a cycle if toxicity occurs and the patient does not recover within 3 days after the planned day of infusion/administration. Administration of the study treatment (isatuximab and/or carfilzomib and/or dexamethasone) is discontinued in the event of an AE that persists despite appropriate dose modifications or any other AE that, in the opinion of the Investigator, warrants discontinuation. All changes to study treatment administration are recorded. Patients receive the next cycle of study treatment after recovery of the toxicity, based on criteria assessed by the investigator.

Dose reduction steps for carfilzomib and dexamethasone are shown in Tables E1 and E2 below, respectively.

TABLE E1
Dose Levels for Carfilzomib Dose Reduction

| Starting dose | | | |
|---|---|---|---|
| (IV) | Dose level -1 | Dose level -2 | Dose level -3 |
| 20 mg/m² | 15 mg/m² | 11 mg/m² | — |
| 56 mg/m² | 45 mg/m² | 36 mg/m² | 27 mg/m² |

TABLE E2
Dose Levels for Dexamethasone Dose Reduction

| Starting dose | | | |
|---|---|---|---|
| (PO/IV) | Dose level -1 | Dose level -2 | Dose level -3 |
| 20 mg | 12 mg | 8 mg | 4 mg |

No dose reductions are allowed for isatuximab infusion.

### V. Disease Assessment

Decisions made by the investigator regarding whether or not to permit subjects to continue treatment were based on efficacy data (obtained from local and/or central laboratories), radiological assessments, and bone marrow assessments performed throughout the study or if indicated according to IMWG criteria. The reference values to assess treatment response were the values measured in samples taken from each patient on Day 1 of Cycle 1, prior to treatment (see Section I. A. Primary Objective above). A summary of assessments and schedule is provided in Table F below. Disease characteristics, including M-protein subtype, extent of bone marrow and extra-medullary disease, cytogenetics (assessed by a central laboratory), and R-ISS, will also be recorded at baseline.

TABLE F
Assessments and Schedule.

| | Timing | Assessment |
|---|---|---|
| Patient | Baseline | Demographics |
| characteristics | | Myeloma history and prior |
| and disease | | anti-myeloma treatment |
| history | | FISH (del[17p], t[4:14], |
| | | t[14:16]) to determine R-ISS |
| | | stage |
| Efficacy | Baseline | Serum M-protein |
| assessment^{†} | Day 1 of all cycles | Urine M-protein |
| | EOT^{‡} | Serum free light chains |
| | Follow-up (for | Quantitative immunoglobulins |
| | patients who | |
| | discontinue the | |
| | study treatment | |
| | without PD) | |
| | Baseline | Bone marrow disease |
| | When indicated to | involvement |
| | document overall | (plasma cell infiltration) |
| | response | |
| | Baseline and then | Bone lytic disease assessment |
| | annually | |
| | Every 12 weeks | Extramedullary disease |
| | (if present at | assessment per investigator |
| | baseline) | choice, with IMWG |
| | | response/failure criteria applied |
| Safety | Continuously | Adverse events |
| | throughout | Vital signs |
| | study period | Physical examination |
| | | Hematology |
| | | Blood chemistry |
| | | Anti-drug antibodies |
| | | Electrocardiogram |
| Isatuximab | Days 1, 8, 15, and | Isatuximab PK parameters by |
| PK | 22 of Cycle 1 | ELISA |
| | Day 1 of subsequent | Population PK modeling |
| | cycles up to 10 cycles | |
| Patient- | Day 1 of all cycles | EORTC QLQ-C30 |
| reported | EOT^{^{‡}} | EORTC QLQ-MY20 |
| outcomes | 90 days after last | EQ-5D-5L |
| | administration | |

^{†}Overall response and disease progression will be assessed by International Myeloma Working Group criteria (Kumar et al 2016).
^{‡}EOT visit will be 30 days after last study treatment administration or before further anti-myeloma therapy initiation, whichever comes first.

ELISA: enzyme-linked immunosorbent assay; EORTC: European Organisation for Research and Treatment of Cancer; EOT: end of treatment; EQ-5D-5L: EuroQoL 5-Dimensions questionnaire with 5 response levels per dimension; FISH: fluorescence in situ hybridization; PD: progressive disease; PK: pharmacokinetics; QLQ-C30: quality of life questionnaire core module; QLQ-MY20: quality of life questionnaire myeloma module; R-ISS: Revised International Staging Score.

The 2016 IMWG criteria (see, e.g., Kumar S, Paiva B, Anderson K C et al. International Myeloma Working Group consensus criteria for response and minimal residual disease assessment in multiple myeloma. The Lancet. Oncology, 17(8), e328-e346 (2016)) are applied to evaluate response and disease progression. Assessments are made on the first day of every cycle and when treatment is stopped. PFS—defined as the time from randomization to either the first documented occurrence of progressive disease or the death of the patient from any cause, whichever occurs earliest—is the primary efficacy endpoint. Response is assessed during follow-up and until disease progression in patients who discontinue therapy prior to disease progression. Subgroup analyses of PFS (e.g., by cytogenetic risk status, number of prior lines of treatment) are also conducted. Next-generation sequencing (NGS) is used to assess MRD.

Safety evaluations include vital signs, hematology and biochemistry assessments, physical examination, electrocardiograms, and AEs: these are followed throughout the study. AEs are graded according to the National Cancer Institute Common Terminology Criteria for AEs v4.03. Immunogenicity is assessed during study treatment. An indirect Coombs test is performed at baseline and after treatment initiation in the isatuximab plus carfilzomib/dexamethasone arm only.

Patient-reported outcome (PRO) assessments are measured on day 1 of every cycle, at the end of treatment, and 90 days after study treatment administration by using the PRO/HRQoL and health utility instruments (European Organisation for Research and Treatment of Cancer quality-of-life questionnaires C30 and MY20 [EORTC QLQ-C30 and QLQMY20] and the EuroQoL questionnaire EQ-5D-5L).

### Example 1B: Initial Results from the Phase III Study Described in Example 1A

302 patients were randomized as follows: 179 patients were assigned to the isatuximab+carfilzomib+dexamethasone (Isa+car+dex) arm, and 123 patients were assigned to the carfilzomib+dexamethasone (car+dex) arm. Patient characteristics were well-balanced across both arms. The median age was 64 years (range: 33-90). 25.8% of the patients were R-ISS Stage I, 59.6% of the patients were R-ISS Stage II, and 7.9% of the patients were R-ISS Stage III. 44% of the patients received 1 prior line of therapy for multiple myeloma, 33% received two prior lines, and 23% received ≥3 prior lines. 90% of the patients had received prior therapy with a proteasome inhibitor, and 78% of the patient had received prior therapy with an immunomodulatory drug (i.e., IMiD®). The median number of prior lines was 2. 24% of the patients had high-risk cytogenetics (i.e., one or more of the following chromosomal/cytogenetic abnormalities: del(17p), t(4;14), and t(14;16)).

As discussed in Example 1A, patients in the experimental arm received isatuximab through an intravenous infusion at a dose of 10 mg/kg once weekly for four weeks, then every other week for 28-day cycles in combination with carfilzomib twice weekly at the 20/56 mg/m² dose and dexamethasone at the standard dose for the duration of treatment. Patients in the control arm received carfilzomib twice weekly at the 20/56 mg/m² dose and dexamethasone at the standard dose for the duration of treatment. The primary endpoint of this study was progression-free survival. Secondary endpoints included overall response rate (ORR), the rate of very good partial response or greater (≥VGPR1, minimal residual disease (MRD), complete response rate (CR), overall survival (OS), and safety.

At a median follow-up of 20.7 months and with 103 progression-free survival (PFS) events per IRC (independent review committee), median PFS was not reached in the isa+car+dex arm. Median PFS in the car+dex arm was 19.15 months (HR 0.531 (99% CI 0.318-0.889), one-sided p=0.0007). Thus, the pre-specified efficacy boundary (p=0.005) was crossed. PFS benefit was consistent across subgroups. The overall response rate (ORR) (i.e., the % of patients achieving partial response (PR) or better) was 86.6% in the isa+car+dex arm vs. 82.9% in the car+dex arm (one-sided p=0.1930). 72.6% of patients in the isa+car+dex arm achieved ≥VGPR (very good partial response), as compared to 56.1% of patients in the car+dex arm (p=0.0011). 39.7% of patients in the isa+car+dex arm achieved complete response (CR) vs. 27.6% of patients in the car+dex arm. MRD negativity rate (10⁻⁵) in intent-to-treat population was 29.6% (53/179) in the isa+car+dex arm vs 13.0% (16/123) in the car+dex arm.

52.0% of the patients in the isa+car+dex arm remained on treatment vs. 30.9% of the patients in the car+dex arm. The main reasons for treatment discontinuation were disease progression (29.1% in the isa+car+dex arm vs. 39.8% in the car+dex arm) and adverse events (8.4% in the isa+car+dex arm vs. 13.8% in the car+dex arm). Grade >3 treatment-emergent adverse effects (TEAEs) were observed in 76.8% of the patients in the isa+car+dex arm vs. 67.2% of the patients in the car+dex arm. Treatment-emergent serious adverse events (TE-SAEs) and fatal TEAEs were similar in both arms: 59.3% of the patients in the isa+car+dex arm experienced TE-SAEs vs 57.4% of the patients in the car-dex arm; and 3.4% of the patients in the isa+car+dex arm experienced fatal TEAEs vs. 3.3% of the patients in the car+dex arm. Infusion reactions were reported in 45.8% (0.6% grade 3-4) of the patients in the isa+car+dex arm and 3.3% (0% grade 3-4) of the patients in the car+dex arm. Grade >3 respiratory infections (grouping) were seen in 32.2% of the patients in the isa+car+dex arm vs. 23.8% of the patients in the car+dex arm. Grade >3 cardiac failure (grouping) was reported in 4.0% of the patients in the isa+car+dex arm vs 4.1% of the patients in the car+dex arm. As per lab results, grade 3-4 thrombocytopenia was reported in 29.9% of the patients in the isa+car+dex arm vs. 23.8% of the patients in the car+dex arm; neutropenia was reported in 19.2% of the patients in the isa+car+dex arm vs 7.4% of the patients in the car+dex arm.

Addition of isatuximab to carfilzomib+dexamethasone provided a superior, statistically significant improvement in PFS with clinically meaningful improvement in depth of response (i.e., MRD) compared to standard-of-care carfilzomib+dexamethasone (i.e., without isatuximab) in patients with relapsed multiple myeloma. Addition of isatuximab to carfilzomib+dexamethasone significantly reduced the risk of disease progression or death compared to standard of care carfilzomib+dexamethasone (i.e., without isatuximab). The isatuximab+carfilzomib+dexamethasone combination was well tolerated with manageable safety and a favorable benefit-risk profile. No new safety signals were identified in this study.

### Example 1C: Further Results from the Phase III Study Described in Example 1A

Further details regarding the Phase III clinical trial described in Example 1A and interim results from the trial are provided in this Example.

Patients with relapsed multiple myeloma who met the inclusion and exclusion criteria described in Example 1A were randomized into the two arms of the study as follows: For every three patients randomized to the IKd arm (isatuximab+carfilzomib+dexamethasone), 2 were randomized to the Kd arm (carfilzomib+dexamethasone). See Table D above. All patients had undergone between 1-3 prior lines of treatment for multiple myeloma. None of the patients received prior therapy with carfilzomib. None of the patients were refractory to prior anti-CD38 therapy. Patients were stratified according to prior lines of therapy (i.e., 1 prior line vs. >1) and R-ISS score (i.e., I or II vs. III vs. not classified) to ensure equal allocation of subgroups of participants to each arm. (Further details about the R-ISS are described in See Palumbo A, et al. Revised International Staging System for Multiple Myeloma: A Report From International Myeloma Working Group. J Clin Oncol. 2015; 33(26):3863-9.)

Key patient demographics and baseline characteristics are shown in Table G below. Patient characteristics were balanced in both arms. For cytogenetic analyses, del 17p was defined using a cut off of 50%, and t(4;14) and 4(14;16) were defined using a cut off of 30%. 3 patients in the IKd arm (1.7%) and 2 patients in the Kd arm (1.6%) received >3 prior lines of therapy for multiple myeloma.

TABLE G
Patient Demographics and Baseline Characteristics

| | Isa-Kd | | Kd | |
|---|---|---|---|---|
| ITT population | (n = 179) | | (n = 123) | |
| Age in years, median (range) | 65.0 | (37-86) | 63.0 | (33-90) |
| Age in years, by category, n (%) | | | | |
| <65 | 88 | (49.2) | 66 | (53.7) |
| 65-<75 | 74 | (41.3) | 47 | (38.2) |
| ≥75 | 17 | (9.5) | 10 | (8.1) |
| CrCl <60 mL/min/1.73 m² | 43 | (26.1) | 18 | (16.2) |
| (MDRD)*, n (%) | | | | |
| ISS stage at baseline, n (%) | | | | |
| Stage I | 89 | (48.7) | 71 | (57.7) |
| Stage II | 63 | (35.2) | 31 | (25.2) |
| Stage III | 26 | (14.5) | 20 | (16.3) |
| Cytogenetic risk at baseline^{†}, % | | | | |
| High | 42 | (23.5) | 31 | (25.2) |
| Standard | 114 | (63.7) | 78 | (63.4) |
| Missing | 23 | (12.8) | 14 | (11.4) |
| Prior lines of therapy, median | 2 | (1-4) | 2 | (1-4) |
| (range)^{†} | | | | |
| 1, n (%) | 79 | (44.1) | 55 | (44.7) |
| 2, n (%) | 64 | (35.8) | 36 | (29.3) |
| 3, n (%) | 33 | (18.4) | 30 | (24.4) |
| Prior proteasome inhibitors | 166 | (92.7) | 105 | (85.4) |
| Prior IMiDs | 136 | (76.0) | 100 | (81.3) |
| Patients refractory to, n (%) | | | | |
| IMiD | 78 | (43.6) | 58 | (47.2) |
| Lenalidomide | 57 | (31.8) | 42 | (34.1) |
| PI | 56 | (31.3) | 44 | (35.8) |
| Last regimen | 89 | (49.7) | 73 | (59.3) |

CrCl, creatinine clearance; d, dexamethasone; IMiD, immunomodulatory drug; Isa, isatuximab; ITT, intent to treat; K, carfilzomib, MDRD, modification of diet in renal disease; ISS, international staging system; PI, proteasome inhibitor.

Treatment continued until patients demonstrated progressive disease (PD), experienced unacceptable toxicities, or elected to leave the study,

The primary endpoints of the study included progression-free survival (PFS), as assessed by an independent review committee (IRC). Secondary endpoints of the study included overall response rate (ORR), rate of ≥very good partial response (VGPR), minimal residual disease (MRD) negativity; complete response (CR) rate, and overall survival (OS).

### Results

At a 20.7-month follow-up, the patient disposition was as follows: Among the 179 patients in the IKd arm, 177 were treated. 84 (46.9%) patients in the IKd arm discontinued treatment. 52 (29.1%) discontinued due to progressive disease (PD); 15 (8.4%) discontinued due to adverse events (AE), and 6 (3.4%) discontinued for other reasons. 93 (52%) patients in the IKd arm remained on treatment. Among the 123 patients in the Kd arm, 122 were treated. 84 (68.3%) patients in the Kd arm discontinued treatment. 49 (39.8%) discontinued due to progressive disease (PD); 17 (13.8%) discontinued due to adverse events (AE), and 4 (3.3%) discontinued for other reasons. 38 (30.9%) patients in the Kd arm remained on treatment. A higher percentage of patients in the IKd arm remained on treatment as compared to the Kd arm (i.e., ˜37% patients in the IKd arm discontinued treatment due to PD or AE, as compared to −54% in the Kd arm).

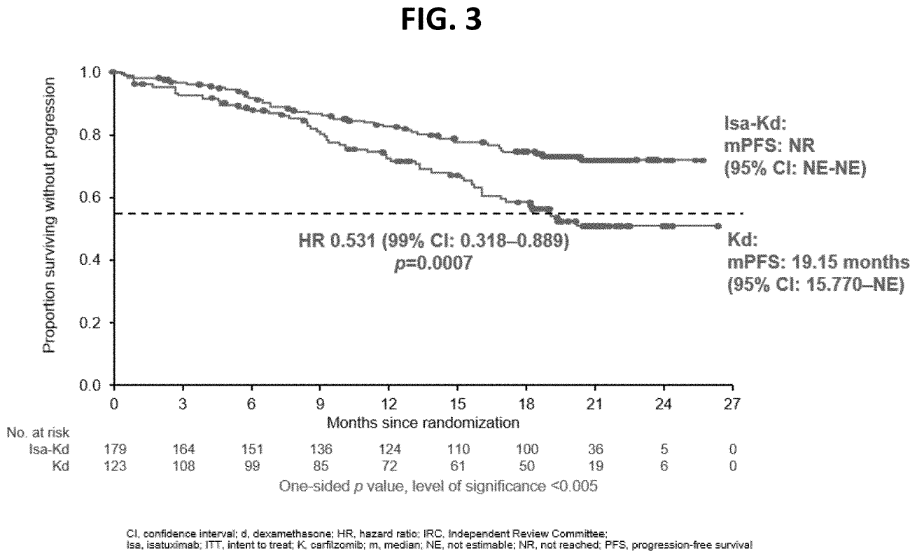

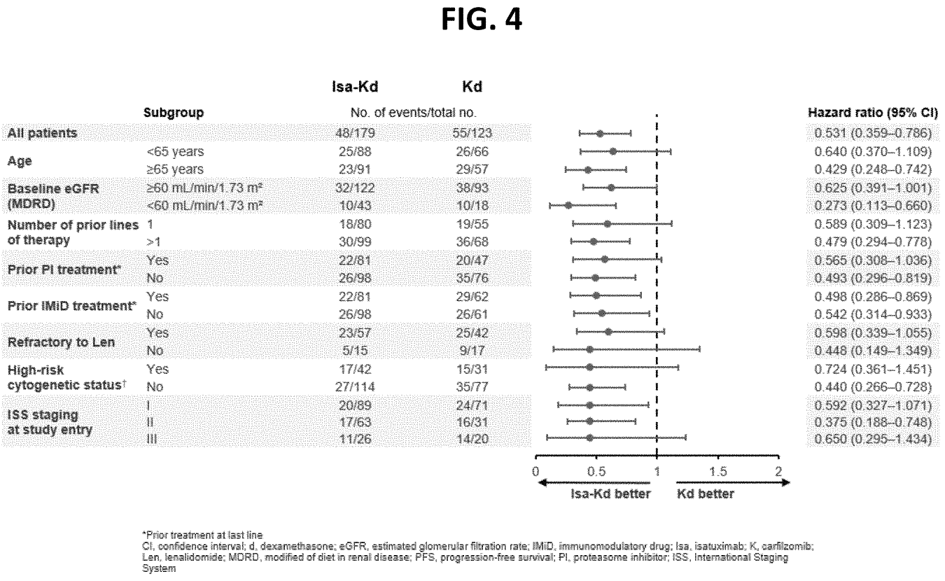

An interim PFS analysis by an independent review committee (IRC) indicated that median PFS (mPFS) had not yet been reached in the IKd arm, whereas the mPFS in the Kd arm was 19.15 months (95% CI: 15.770-NE). HR 0.531 (99% CI: 0.318-0.889), p=0.0007. Patients receiving IKd showed improvement in PFS, with a 47% reduction in the risk of disease progression or death, as compared to patients in the Kd arm. See FIG. 3. Subgroup analyses were performed for PFS. As shown in FIG. 4, all subgroups favored IKd vs. Kd. A consistent treatment effect was seen for IKd across all subgroups analyzed (e.g., age, baseline kidney function (eGFR), number of prior lines of therapy, prior proteasome inhibitor treatment at last line, prior immunomodulatory drug treatment at last line, high-risk cytogenetic status, ISS staging at study entry, and being refractory to lenalidomide).

Deeper responses were seen in patients treated with IKd as compared to those treated with Kd, consistent with PFS improvement. The overall response rate (ORR) of patients in the IKd arm was 86%, whereas the ORR of patients in the Kd arm was 82% (p=0.19, stratified Cochran-Mantel-Haenszel test; one sided significant level is 0.025). 72.6% of the patients in the IKd arm achieved VGPR or better, as compared to 56.1% of the patients in the Kd arm (p=0.0011). 39.7% of the patients in the IKd arm achieved CR, as compared to 27.6% of the patients in the Kd arm. Further, more patients in the IKd arm were MRD-negative (i.e., “minimal residual disease negative” at a threshold of 10⁻⁵, as assessed by next-generation sequencing (NGS)) than in the Kd arm. Among Intent To Treat patients, 53/179 (29.6%) in the IKd arm were MRD-negative, as compared to 16/123 (13%) in the Kd arm. Among patients in the study who achieved VGPR or better, 53/128 (41.4%) in the IKd arm were MRD-negative, as compared to 16/70 (22.9%) in the Kd arm.

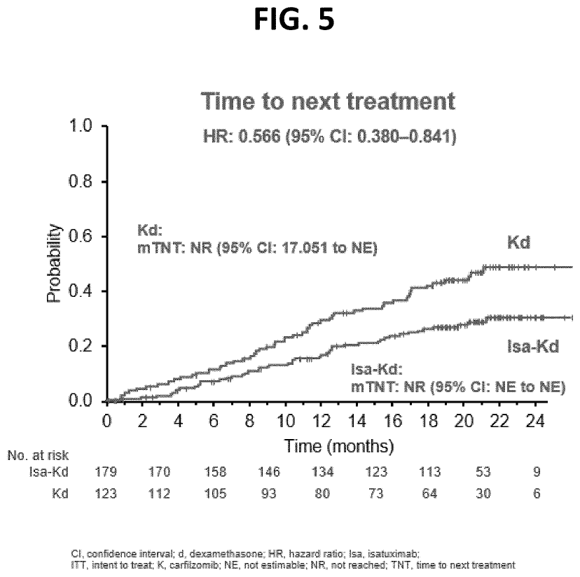

Treatment with IKd resulted in a notable delay in time to next treatment, as compared to treatment with Kd, consistent with PFS improvement. See FIG. 5 and Table H.

TABLE H
Time to Next Treatment.

| | | |
|---|---|---|
| | Isa-Kd | Kd |
| ITT population | (n = 179) | (n = 123) |

Kaplan-Meier estimates of TNT in
months

| | | |
|---|---|---|
| Median (95% Cl) | NR | NR |

| | | | | |
|---|---|---|---|---|
| Patients with further anti-myeloma | 47 | (26.3) | 53 | (43.1) |
| treatment, n (%) | | | | |
| Main treatments, n (%) | | | | |
| Alkylating agents | 26 | (55.3) | 21 | (39.6) |
| Proteasome inhibitors | 16 | (34.0) | 11 | (20.8) |
| Bortezomib | 11 | (23.4) | 9 | (17.0) |
| Carfilzomib | 2 | (4.3) | 1 | (1.9) |
| Ixazomib | 6 | (12.8) | 1 | (1.9) |
| Immunomodulators | 39 | (83.0) | 42 | (79.2) |
| Lenalidomide | 19 | (40.4) | 23 | (43.4) |
| Pomalidomide | 24 | (51.1) | 21 | (39.6) |
| Thalidomide | 5 | (10.6) | 4 | (7.5) |
| Monoclonal antibodies | 11 | (23.4) | 29 | (54.7) |
| Daratumumab | 10 | (21.3) | 25 | (47.2) |
| Further transplant | 6 | (12.8) | 5 | (9.4) |

At a 20.73-month follow up, overall survival (OS) data was not mature at the time of analysis.

Exposure to study treatments in each treatment arm are shown in Table I. The high relative dose intensity of both isatuximab and carfilzomib in the IKd arm demonstrates the feasibility of the combination.

TABLE I
Exposure to Study Treatments.

| | Isa-Kd | | Kd | |
|---|---|---|---|---|
| Safety population | (n = 177) | | (n = 122) | |
| Median treatment duration, | 80 | (1-111) | 61.4 | (1-114) |
| weeks (range) | | | | |
| Relative dose intensity, | | | | |
| median (range) | | | | |
| Isatuximab | 94.27 | (66.7-108.2) | — | |
| Carfilzomib | 91.18 | (18.2-108.7) | 91.35 | (41.8-108.6) |
| Dexamethasone | 84.78 | (24.5-101.1) | 88.37 | (27.4-101.6) |
| Total number of cycles | 2813 | | 1663 | |
| Cycle delayed, n (%) | 304 | (10.8) | 160 | (9.6) |
| Between 4 and 7 days | 176 | (6.3) | 96 | (5.8) |
| More than 7 days | 128 | (4.6) | 64 | (3.8) |

More patients in the IKd experienced Grade >3 treatment-emergent adverse events (TEAEs) than in the Kd arm (76.8% IKd vs. 67.2% Kd). The addition of isatuximab to carfilzomib to carfilzomib+dexamethasone did not increase mortality, serious TEAEs, or events leading to discontinuation of treatment. IKd has a manageable safety profile with no new safety signals. Infusion reactions (IRs) mainly occurred during the first infusion and were mostly Grade 1 or 2.

### Conclusions

Addition of Isatuximab to Kd resulted in a statistically significant improvement in PFS with HR of 0.531, corresponding to a 47% reduction in the risk of progression or death. IKd showed a consistent benefit across multiple subgroups, including those difficult to treat with high unmet medical need (elderly, high-risk cytogenetic, renally impaired). IKd showed a profound depth of response compared to Kd, with an MRD negativity rate of 30% vs 13% in the ITT population. IKd demonstrated a manageable safety profile and favorable risk/benefit in patients with relapsed MM.

### Example 1D: Depth of Response and Response Kinetics of Isatuximab Plus Carfilzomib and Dexamethasone in Relapsed Multiple Myeloma

### Introduction

Achievement of minimal residual disease negative (MRD−) status in multiple myeloma (MM) is associated with improved progression-free survival (PFS) and overall survival (OS). Isatuximab (Isa) is an approved anti-CD38 IgG kappa monoclonal antibody. The depth of response including MRD−, long-term outcomes, and kinetics of tumor response in study described in Example 1A were analyzed. Measurement by mass spectrometry of serum M-protein was also performed to overcome the interference with Isa in standard immunofixation assay.

### Methods

Example 1A describes a randomized, open-label, multicenter Phase 3 study that investigated Isa plus carfilzomib and dexamethasone (Isa-Kd) vs. Kd in patients with relapsed MM who received 1-3 lines of therapy. The primary endpoint of PFS and secondary endpoints of overall response rate (ORR), very good partial response or better (≥VGPR) and complete response (CR) rate were determined by an Independent Response Committee (IRC) based on central data for M-protein, central imaging review and local bone marrow for plasma cell infiltration according to International Myeloma Working Group (IMWG) criteria (see, e.g., in Kumar et al. (2016) “International Myeloma Working Group consensus criteria for response and minimal residual disease assessment in multiple myeloma.” Lancet Oncol. 17(8): e328-e346) and Dune et al. (2006) “International uniform response criteria for multiple myeloma. Leukemia. 20: 1467-1473). MRD (i.e., minimal residual disease) was assessed in bone marrow aspirates from patients who achieved ≥VGPR by next generation sequencing at 10⁻⁵ sensitivity level. Mass spectrometry analysis was performed to measure serum M-protein without Isa interference. Hazard ratios and corresponding confidences interval were estimated using Cox proportional hazards model. Secondary endpoints were compared between treatment arms using Cochran Mantel Haenszel test. All randomized patients not reaching MRD- or without MRD assessment were analyzed as MRD+.

### Results

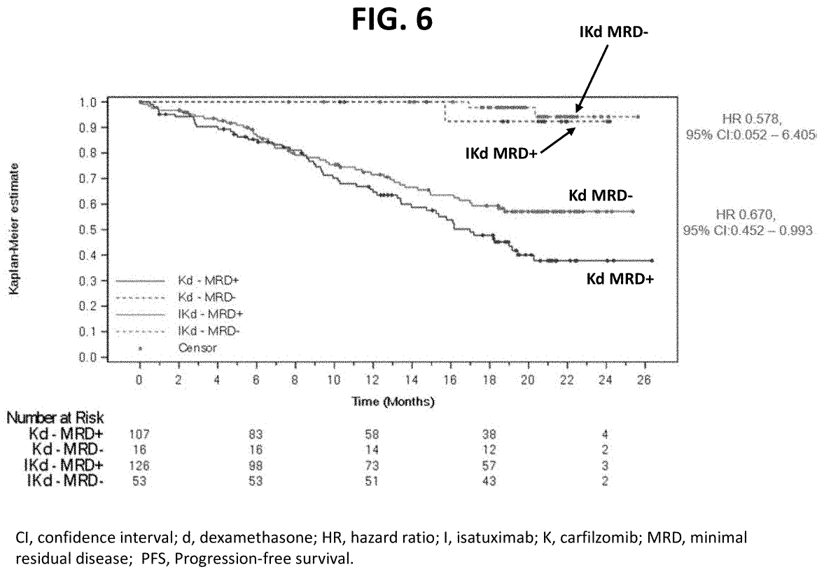

As discussed in Example 1A, 302 patients (179 Isa-Kd, 123 Kd) were randomized. At a median follow-up of 20.7 months, deeper responses were observed in patients receiving Isa-Kd than in those receiving Kd. 72.6% of the patients in the Isa-Kd arm achieved ≥VGPR vs. 56.1% of patients in the Kd arm (nominal p=0.011). 39.7% of patients in the Isa-kd arm achieved and ≥CR vs. 27.6% of the patients in the Kd arm. MRD− occurred in 53/179 (30%) of the patients in the Isa-Kd arm vs. 16/123 (13%) of patients in the Kd arm (nominal p=0.0004). (See also, e.g., Examples 1B and 1C). 20.1% (36/179) of the patients in the Isa-Kd arm achieved both CR and MRD−, as compared to 10.6% (13/123) patients in the Kd arm. Progression free survival (PFS) by MRD status in both treatment arms (i.e., Isa-Kd vs. Kd) is shown in FIG. 6. The hazard ratio (HR) favors Isa-Kd, rather than Kd, in both MRD-patients (HR 0.578, 95% CI: 0.052-6.405) and MRD+ patients (HR 0.670, 95% CI: 0.452-0.993). MRD-patients had a longer PFS than MRD+ patients. Within the Isa-Kd arm, MRD-negative status could be obtained in patients with renal impairment, i.e., with eGFR <60 mL/min/1.73 m², (26.5% MRD− vs 25.9% MRD+); with ISS stage III at diagnosis (32.1% MRD− vs 27.8% MRD+); with t(4;14) [13.2% MRD− vs 11.9% MRD+], with gain(1q21) [45.3% MRD− vs 40.5% MRD+]; in heavily pretreated ≥3 prior lines (22.6% MRD− vs 19.0% MRD+) or refractory to lenalidomide in last regimen (18.9% MRD− vs 20.6% MRD+). Within the Isa-Kd arm, MRD-negative status was reached less frequently in patients refractory to a proteasome inhibitor (PI) [18.9% MRD-vs 36.5% MRD+) or with del(17p), [3.8% MRD− vs 12.7% MRD+].

Interference of isatuximab with M protein was explored: samples from 27 patients with near-CR (only serum immunofixation (IF) positive IgG kappa) or potential CR (serum remaining M-protein <0.5 g/dL with IF positive IgG kappa) in the Isa-Kd arm were tested by mass spectrometry. Among them, 11 near CR or potential CR pts had documented <5% plasma cells in bone marrow and were mass spectrometry negative (residual myeloma M-protein level below limit of quantification (LOQ) of central lab immunofixation). In addition, of the 11 near CR or potential CR patients, 7 were also MRD−. These results support that both current CR rate and MRD− CR rate are underestimated (potential adjusted CR rate of 45.8%; potential adjusted MRD− CR rate 24%).

Responses to treatment occurred quickly in both arms. The median time in responders to first response was 32.0 (28-259) days in the Isa-Kd arm vs 33.0 (27-251) days in the Kd arm. The median time in responders to best response was 120.0 (29-568) days in the Isa-Kd vs 104.5 (29-507) days in the Kd arm. The median time in responders to first CR was 184.0 (30-568) days in the Isa-Kd arm vs 229.5 (58-507) days in the Kd arm. The median time in responders to first ≥VGPR was 88.0 (28-432) days in the Isa-Kd arm vs 90.0 (29-491) days in the Kd arm. In addition to increased depth of response, quality of life as measured by the European Organization for Research and Treatment of Cancer (EORTC) Quality-of-Life Questionnaire-C30 Global Health Status scores was maintained in patient treated with Isa-Kd per descriptive analyses.

### Conclusions

There was a clinically meaningful improvement in depth of response in patients treated with Isa-Kd vs. patients treated with Kd. The CR rate of 39.7% in the Isa-Kd arm of the study as underestimated due to interference. Mass spectrometry results suggest that CR could be reached for approximately half of the patients with 1 to 3 prior line treated with Isa-Kd. More patients in Isa-Kd arm than in the Kd arm reached MRD negativity (30% vs 13%), and at least twice as many patients in the Isa Kd arm reached CR MRD− that in the Kd arm (20.1% vs 10.6%; adjusted 24% vs 10.6%, respectively). Reaching MRD negativity was associated with longer PFS in both arms.

### Example 1E: Further Results from the Phase III Study Described in Example 1A

Eligible patients had relapsed and/or refractory multiple myeloma with one to three prior lines of therapy and measurable evidence of disease (serum M-protein ≥0.5 g/dl and/or urine M-protein ≥200 mg/24 hours). Patients were excluded if they had primary refractory multiple myeloma, per International Myeloma Working Group (IMWG) response criteria (see, e.g., in Kumar et al. (2016) “International Myeloma Working Group consensus criteria for response and minimal residual disease assessment in multiple myeloma.” Lancet Oncol. 17(8): e328-e346) and Durie et al. (2006) “International uniform response criteria for multiple myeloma. Leukemia. 20: 1467-1473), serum-free light chain measurable disease only, or Eastern Cooperative Oncology Group performance status >2. Patients were excluded if they received anti-myeloma treatment within 14 days of randomization, prior treatment with carfilzomib, were refractory to anti-CD38 antibody therapy, or had a contraindication to dexamethasone. Patients with estimated glomerular filtration rate (eGFR) according to the modification of diet in renal disease formula <15 ml/min/1.73 m² or left ventricular ejection fraction <40% were also excluded. Patients with prior pulmonary comorbidities, including chronic obstructive pulmonary disease, were enrolled. Patients were randomized as discussed above, and randomization was stratified as discussed above. Patients in each arm were treated as outlined in Table D.

### Efficacy Endpoints and Assessments

The primary efficacy end point was progression-free survival, as per a blinded independent response committee (IRC). The IRC reviewed disease assessments for response and progression (central radiological evaluation, M-protein quantification from central lab and local bone marrow aspiration for plasma cell infiltration when needed). Key secondary efficacy end points included overall response rate according to IMWG response criteria, very good partial response (VGPR) or better rate, minimal residual disease (MRD) negativity rate, complete response (CR) rate, and overall survival.

MRD was assessed by next-generation sequencing with a minimum sensitivity of 1 in 10⁵ nucleated cells in patients reaching ≥VGPR. Cytogenetics was assessed by fluorescence in situ hybridization (FISH) during screening by a central laboratory, with a cutoff of 50% for del(17p) and 30% for t(4;14), t(14;16) and gain(1q21). High-risk cytogenetic status was defined as presence of del(17p), t(4;14) or t(14;16).

Efficacy assessments were completed on day 1 of every cycle and when treatment stopped. Safety assessments included recording of adverse events, laboratory parameters (both graded per National Cancer Information Center-Common Terminology Criteria (NCIC-CTC) version 4.03), vital signs, electrocardiograms, and Eastern Cooperative Oncology Group performance status. Efficacy analyses were performed on the intent-to-treat population and summarized by the randomized treatment. Safety analyses and extent of study treatment were assessed and summarized by actual treatment received within the safety population.

### Patients and Treatment

Demographics and clinical characteristics were well balanced at baseline (Table J). Median age was 64 years (range, 33-90). Median number of prior lines was 2 (range, 1-4) and was similar between arms with 44%, 33%, and 23% having received 1, 2, and ≥3 prior lines respectively. One patient (isatuximab arm) and two patients (control arm) received four prior lines. Overall, 45% of patients were refractory to immunomodulatory drugs, including 32.8% lenalidomide-refractory. In the isatuximab arm, 23.5% of patients had high-risk cytogenetics, similar to the control arm (25.2%). At baseline, 26.1% of patients were renally impaired (eGFR <60 ml/min/1.73 m²) in the isatuximab arm versus 16.2% in the control arm.

TABLE J
Demographic, Baseline Disease and Clinical Characteristics
in the Randomized Population.

| | Isa-Kd | | Kd | |
|---|---|---|---|---|
| | (n = 179) | | (n = 123) | |
| Age (years) | | | | |
| Median (range) | 65.0 | (37-86) | 63.0 | (33-90) |
| Age group (years) [n (%)] | | | | |
| <65 | 88 | (49.2) | 66 | (53.7) |
| ≥65 to <75 | 74 | (41.3) | 47 | (38.2) |
| ≥75 | 17 | (9.5) | 10 | (8.1) |
| Gender [n(%)] | | | | |
| Female | 78 | (43.6) | 55 | (44.7) |
| Male | 101 | (56.4) | 68 | (55.3) |
| Race [n(%)] | | | | |
| White | 131 | (73.2) | 83 | (67.5) |
| Black or African American | 5 | (2.8) | 4 | (3.3) |
| Asian | 26 | (14.5) | 24 | (19.5) |
| Other/Not reported | 17 | (9.5) | 12 | (9.8) |
| eGFR (MDRD)*, [n (%)] | | | | |
| <60 ml/min/1.73 m² | 43 | (26.1) | 18 | (16.2) |
| ≥60 ml/min/1.73 m² | 122 | (73.9) | 93 | (83.8) |
| Eastern Cooperative Oncology | | | | |
| Group performance status | | | | |
| [n(%)] | | | | |
| 0 | 95 | (53.1) | 73 | (59.3) |
| 1 | 73 | (40.8) | 45 | (36.6) |
| 2 | 10 | (5.6) | 5 | (4.1) |
| 3 | 1 | (0.6) | 0 | |
| Multiple myeloma subtype at | | | | |
| study entry [n(%)] | | | | |
| Number | 179 | | 123 | |
| Immunoglobulin G | 126 | (70.4) | 85 | (69.1) |
| Immunoglobulin A | 38 | (21.2) | 30 | (24.4) |
| Immunoglobulin D | 4 | (2.2) | 1 | (0-8) |
| Kappa light chain only (urines) | 5 | (2.8) | 4 | (3.3) |
| Lambda light chain only (urines) | 6 | (3.4) | 3 | (2.4) |
| Beta 2-microglobulin (mg/L), | | | | |
| [n(%)] | | | | |
| <3.5 | 103 | (57.5) | 79 | (64.2) |
| ≥3.5 to <5.5 | 50 | (27.9) | 24 | (19.5) |
| ≥5.5 | 26 | (14.5) | 20 | (16.3) |
| Serum LDH (IU/L) [n(%)] | | | | |
| ≤ULN | 132 | (75.0) | 105 | (86.1) |
| >ULN | 44 | (25.0) | 17 | (13.9) |
| Time from initial diagnosis of MM | | | | |
| to randomization (years) | | | | |
| Median (range) | 3.23 | (0.4-17.9) | 3.33 | (0.2-21.3) |
| International Staging System stage | | | | |
| at study entry [n(%)] | | | | |
| Stage I | 89 | (49.7) | 71 | (57.7) |
| Stage II | 63 | (35.2) | 31 | (25.2) |
| Stage III | 26 | (14.5) | 20 | (16.3) |
| Unknown | 1 | (0-6) | 1 | (0-8) |
| Cytogenetic risk as defined for | | | | |
| Revised International Staging | | | | |
| System | | | | |
| High-risk CA† | 42 | (23.5) | 31 | (25.2) |
| Standard risk CA | 114 | (63.7) | 78 | (63.4) |
| Unknown or missing | 23 | (12.8) | 14 | (11.4) |
| Number of prior lines | | | | |
| Median (range) | 2 | (1-4) | 2 | (1-4) |
| Autologous transplant [n(%)] | 116 | (64.8) | 69 | (56.1) |
| Main anti-myeloma therapies by | | | | |
| class and agent [n(%)] | | | | |
| Alkylating agents | 169 | (94.4) | 101 | (82.1) |
| Proteasome inhibitors | 166 | (92.7) | 105 | (85.4) |
| Immunomodulators | 136 | (76.0) | 100 | (81.3) |
| Lenalidomide | 72 | (40.2) | 59 | (48.0) |
| Corticosteroids | 179 | (100) | 123 | (100) |
| Monoclonal antibodies | 5 | (2.8) | 1 | (0.8) |
| Daratumumab | 1 | (0-6) | 0 | |
| Refractory to IMiD [n (%)] | 78 | (43.6) | 58 | (47.2) |
| Refractory to lenalidomide | 57 | (31.8) | 42 | (34.1) |
| Refractory to PI [n (%)] | 56 | (31.3) | 44 | (35.8) |
| Refractory to IMiD and PI | 35 | (19.6) | 27 | (22.0) |
| [n (%)] | | | | |
| Refractory to last regimen | 89 | (49.7) | 73 | (59.3) |
| [n (%)] | | | | |

eGFR, estimated glomerular filtration rate;
IMiD, immunomodulatory imide drug;
Isa-Kd, isatuximab-carfilzomib-dexamethasone;
Kd, carfilzomib-dexamethasone;
LDH, lactate dehydrogenase;
MDRD, modification of diet in renal disease;
PI, proteasome inhibitor;
ULN, upper limit of normal
*Incidence calculated on patients with race reported in case report form: 165 patients in Isa-Kd arm, 111 patients in Kd arm;
†High-risk cytogenetic status is defined as the presence of del(17p) and/or translocation t(4; 14) and/or translocation t(14; 16). Chromosomal Abnormality (CA) was considered positive if present in at least 30% of analyzed plasma cells, except for del(17p) where the threshold is at least 50%.

At the time of analysis, median treatment duration was 80.0 weeks (range, 1-111) in the isatuximab arm and 61.4 weeks (range 1-114) in the control arm. Median relative dose intensity of carfilzomib and dexamethasone were similar in both arms (91.2% and 84.8% for the isatuximab arm vs. 91.4% and 88.4% for the control arm, respectively). The median relative dose intensity of isatuximab was 94.3%. Fewer patients (46.9% vs. 68.3%) discontinued treatment in the isatuximab versus control arm.

### Efficacy

At a median follow-up of 20.7 months, the addition of isatuximab to carfilzomib-dexamethasone demonstrated a statistically significant improvement in progression-free survival with a hazard ratio of 0.531 (99% CI, 0.318-0.889, one-sided P value=0.0007), corresponding to a 46.9% reduction in the risk of progression or death. The median progression-free survival of 19.15 months (95% CI, 15.770—not reached) in the Kd was consistent with the protocol assumption of 19 months. Median PFS was not reached in the IKd arm. At two years, the progression-free survival probability was 68.9% (IKd arm) versus 45.7% (Kd arm).

In the intent-to-treat population, the overall response rate was 86.6% (IKd arm) versus 82.9% (Kd arm), one-sided P value=0.1930. The difference between arms was not statistically significant, thus P values of subsequent key secondary end points are provided for descriptive purposes only. VGPR or better rate was 72.6% (IKd arm) versus 56.1% (Kd arm) (P=0.0011). The CR rate was 39.7% (IKd arm) versus 27.6% (Kd arm). The MRD negativity rate more than doubled in the intent-to-treat population by the addition of isatuximab to carfilzomib-dexamethasone: 29.6% (IKd arm) versus 13.0% (Kd arm) (P=0.0004) (Table K). The proportion of patients achieving both CR and MRD-negative response was 20.1% (IKd arm) and 10.6% (Kd arm). While overall survival was not mature at the interim analysis, 17.3% and 20.3% of patients died in the isatuximab and control arms, respectively.

TABLE K
Summary of Responses in the Intent-to-Treat (ITT) Population.

| | Isa-Kd | | Kd | |
|---|---|---|---|---|
| | (n = 179) | | (n = 123) | |
| Best Overall Response [n (%)] | | | | |
| Stringent complete response | 0 | | 0 | |
| Complete response | 71 | (39.7) | 34 | (27.6) |
| Very good partial response | 59 | (33.0) | 35 | (28.5) |
| Biochemical complete response | 6 | (3.4) | 7 | (5.7) |
| but with missing bone marrow† | | | | |
| Near-complete response‡ | 36 | (20.1) | 13 | (10.6) |
| Partial response | 25 | (14.0) | 33 | (26.8) |
| Minimal response | 4 | (2.2) | 5 | (4.1) |
| Stable disease | 13 | (7.3) | 6 | (4.9) |
| Non-progressive disease | 1 | (0.6) | 1 | (0.8) |
| Progressive disease | 2 | (1.1) | 3 | (2.4) |
| Unconfirmed progressive disease | 0 | | 1 | (0.8) |
| Not evaluable/Not assessed | 4 | (2.2) | 5 | (4.1) |
| Overall Response | | | | |
| Responders (stringent complete | 155 | (86.6) | 102 | (82.9) |
| response, complete response, very | | | | |
| good partial response, or partial | | | | |
| response) | | | | |
| 95% confidence interval§ | 0.8071 to 0.9122 | | 0.7509 to 0.8911 | |
| Stratified | | | | |
| Cochran-Mantel_Haenszel test P | | | | |
| value∥ | | | | |
| vs Kd | 0.1930 | | | |
| Very good partial response or | 130 | (72.6) | 69 | (56.1) |
| better | | | | |
| 95% confidence interval∥ | 0.6547 to 0.7901 | | 0.4687 to 0.6503 | |
| Stratified | | | | |
| Cochran-Mantel-Haenszel test P | | | | |
| value∥ | | | | |
| vs Kd | 0.0011 | | | |
| Minimal residual disease | 53 | (29.6) | 16 | (13.0) |
| negativity rate¶ | | | | |
| 95% confidence interval∥ | 0.2303 to 0.3688 | | 0.0762 to 0.2026 | |
| Stratified | | | | |
| Cochran-Mantel-Haenszel test P | | | | |
| value∥ | | | | |
| vs Kd | 0.0004 | | | |
| Complete response rate (stringent | 71 | (39.7) | 34 | (27.6) |
| complete response or complete | | | | |
| response) | | | | |
| 95% confidence interval∥ | 0.3244 to 0.4723 | | 0.1996 to 0.3643 | |
| Minimal residual disease | 36 | (20.1) | 13 | (10.6) |
| negativity and complete | | | | |
| response (stringent complete | | | | |
| response or complete response) | | | | |

| | | |
|---|---|---|
| 95% confidence interval∥ | 0.1450 to 0.2674 | 0.0575 to 0.1740 |

Isa-Kd: isatuximab-carfilzomib-dexamethasone; Kd: carfilzomib-dexamethasone.
†Two consecutive negative M-protein and negative immunofixation with missing bone marrow
‡All criteria for a complete response were met except that immunofixation remained positive
§Estimated using Clopper-Pearson method.
∥Stratified on randomization factors according to interactive response technology. One-sided significance level is 0.025.
Biochemical complete response and near-complete response were assessed only for patients with confirmed very good partial response as best overall response. Criteria for confirmation was not applied to near-complete response subcategory.
¶For analysis purposes, subjects in the intent-to-treat population but without minimal residual disease assessment will be considered as having positive minimal residual disease.

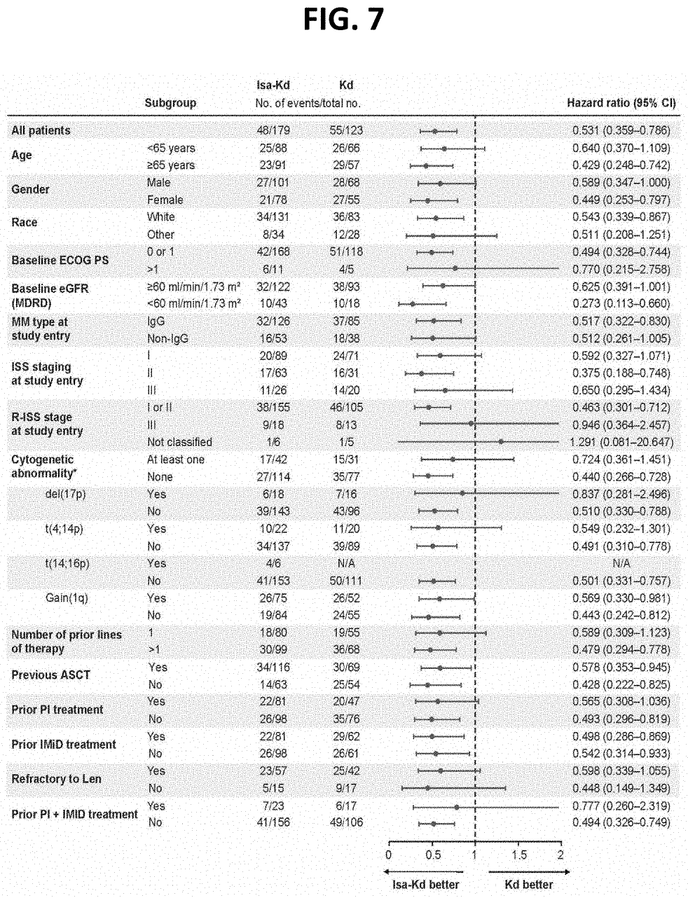

In the pre-specified subgroup analyses, clinical benefit in favor of isatuximab with carfilzomib-dexamethasone occurred across almost all groups (FIG. 7). Median progression-free survival in renally impaired patients was not reached in the IKd arm versus 13.41 months (95% CI, 4.830—not reached) in the Kd arm with a hazard ratio of 0.273 [95% CI, 0.113-0.660]. Complete renal response (improvement in eGFR <50 ml/min/1.73 m² at baseline to ≥60 ml/min/1.73 m²) occurred in 52% (IKd arm) versus 30.8% (Kd arm) and was durable in 32.0% (IKd arm) versus 7.7% (Kd arm) of patients, respectively. Progression-free survival benefit in favor of isatuximab with carfilzomib-dexamethasone was observed in elderly patients (?65 years old), including a hazard ratio of 0.244 (95% CI, 0.060-1.000) for those ≥75 years of age.

Median time to first response in responders was similar in both arms: 32 days (IKd arm) and 33 days (Kd arm); duration of response was longer in the IKd arm with a hazard ratio of 0.425 (95% CI, 0.269 to 0.672). Isatuximab plus carfilzomib-dexamethasone delayed time to next treatment (hazard ratio, 0.566; 95% CI, 0.380-0.841). 26.3% of patients (IKd arm) received at least one further anti-myeloma therapy versus 43.1% (Kd arm), and of those who received subsequent treatment, 21.3% and 47.2% received daratumumab, respectively. Health-related quality of life, as measured by QLQ-C30 Global Health Status score, was maintained with isatuximab plus carfilzomib-dexamethasone.

### Discussion

The results of this randomized, Phase 3 study showed that addition of isatuximab to carfilzomib-dexamethasone was associated with a significant benefit in progression-free survival in patients with relapsed multiple myeloma versus carfilzomib-dexamethasone alone. The risk of disease progression or death was 47% lower in the isatuximab arm, indicated by the very low hazard ratio (0.531 [99% CI, 0.318-0.889]). The median progression-free survival of 19.15 months in the control arm was consistent with the protocol assumption (19 months) and a prior Phase 3 trial assessing the efficacy of carfilzomib plus dexamethasone versus bortezomib plus dexamethasone in patients with relapsed/refractory multiple myeloma after one to three prior lines. The present results indicate that superiority of the IKd arm was not related to a poorly performing control arm (i.e., Kd arm).

A benefit in progression-free survival was seen in almost all subgroups in the IKd arm, including high-risk cytogenetics, International Staging System stage III at study entry, elderly patients, patients with renal impairment, patients with ≥1 prior line of therapy, prior exposure to an immunomodulatory drug, prior exposure to a proteasome inhibitor, and prior exposure to both an immunomodulatory drug and proteasome inhibitor. Of importance, cytogenetic risk was assessed centrally for all patients using internationally accepted cut-offs for FISH positivity and was conclusive for 88% of patients overall.

The depth and quality of response was better in the IKd arm versus the Kd arm, with higher rates of VGPR, CR, MRD negativity, and CR with MRD negativity. Specifically, the rates of MRD negativity and CR with MRD negativity in the IKd arm were very high considering these patients had a median of two prior lines. Additionally, the rate of CR with MRD negativity is likely underestimated because CR was assessed without use of an interference assay (see, e.g., Example 1D).

In this study conducted in patients with relapsed multiple myeloma, the addition of isatuximab to carfilzomib-dexamethasone resulted in significantly longer progression-free survival compared to carfilzomib-dexamethasone alone. The depth and quality of response was better in the isatuximab arm, including a high CR with MRD negativity rate, which is a prognostic factor for better progression-free survival and overall survival. The safety profile was manageable and expected, with no increase in cardiovascular events. Taken together, these results demonstrate that isatuximab plus carfilzomib-dexamethasone represents a potential new standard of care for patients with relapsed multiple myeloma.

Each embodiment herein described may be combined with any other embodiment or embodiments unless clearly indicated to the contrary. In particular, any feature or embodiment indicated as being preferred or advantageous may be combined with any other feature or features or embodiment or embodiments indicated as being preferred or advantageous, unless clearly indicated to the contrary.

All references cited in this application are expressly incorporated by reference herein.

1. An anti-CD38 antibody comprising (a) a heavy chain variable domain (V_{H}) that comprises: a CDR-H1 comprising the amino acid sequence DYWMQ (SEQ ID NO: 1), a CDR-H2 comprising the amino acid sequence TIYPGDGDTGYAQKFQG (SEQ ID NO: 2), and a CDR-H3 comprising the amino acid sequence GDYYGSNSLDY (SEQ ID NO: 3), and (b) a light chain variable domain (V_{L}) that comprises: a CDR-L1 comprising the amino acid sequence KASQDVSTVVA (SEQ ID NO: 4), a CDR-L2 comprising the amino acid sequence SASYRYI (SEQ ID NO: 5), and a CDR-L3 comprising the amino acid sequence QQHYSPPYT (SEQ ID NO: 6) for use in a method of treating multiple myeloma in an individual, the method comprising administering the anti-CD38 antibody, carfilzomib, and dexamethasone to the individual,
wherein the anti-CD38 antibody is administered at a dose of 10 mg/kg, the carfilzomib is administered at a dose of 20 mg/m² or 56 mg/m², and the dexamethasone is administered at a dose of 20 mg,
wherein the individual received at least one prior therapy for multiple myeloma, and wherein the treatment extends progression free survival (PFS) and/or overall survival (OS) of the individual.

2. An anti-CD38 antibody comprising (a) a heavy chain variable domain (V_{H}) that comprises: a CDR-H1 comprising the amino acid sequence DYWMQ (SEQ ID NO: 1), a CDR-H2 comprising the amino acid sequence TIYPGDGDTGYAQKFQG (SEQ ID NO: 2), and a CDR-H3 comprising the amino acid sequence GDYYGSNSLDY (SEQ ID NO: 3), and (b) a light chain variable domain (V_{L}) that comprises: a CDR-L1 comprising the amino acid sequence KASQDVSTVVA (SEQ ID NO: 4), a CDR-L2 comprising the amino acid sequence SASYRYI (SEQ ID NO: 5), and a CDR-L3 comprising the amino acid sequence QQHYSPPYT (SEQ ID NO: 6) for use in a method of treating multiple myeloma in an individual, the method comprising the method comprising administering the anti-CD38 antibody, carfilzomib, and dexamethasone to the individual,
wherein the anti-CD38 antibody is administered at a dose of 10 mg/kg, the carfilzomib is administered at a dose of 20 mg/m² or 56 mg/m², and the dexamethasone is administered at a dose of 20 mg,
wherein the individual received at least one prior therapy for multiple myeloma, and
wherein the individual is Minimal Residual Disease negative at a threshold of 10⁻⁵ or less after treatment.

3. An anti-CD38 antibody comprising (a) a heavy chain variable domain (V_{H}) that comprises: a CDR-H1 comprising the amino acid sequence DYWMQ (SEQ ID NO: 1), a CDR-H2 comprising the amino acid sequence TIYPGDGDTGYAQKFQG (SEQ ID NO: 2), and a CDR-H3 comprising the amino acid sequence GDYYGSNSLDY (SEQ ID NO: 3), and (b) a light chain variable domain (V_{L}) that comprises: a CDR-L1 comprising the amino acid sequence KASQDVSTVVA (SEQ ID NO: 4), a CDR-L2 comprising the amino acid sequence SASYRYI (SEQ ID NO: 5), and a CDR-L3 comprising the amino acid sequence QQHYSPPYT (SEQ ID NO: 6) for use in a method of treating multiple myeloma in an individual, the method comprising administering the anti-CD38 antibody, carfilzomib and dexamethasone to the individual,
wherein the anti-CD38 antibody is administered at a dose of 10 mg/kg, the carfilzomib is administered at a dose of 20 mg/m² or 56 mg/m², and the dexamethasone is administered at a dose of 20 mg,
wherein the individual received at least one prior therapy for multiple myeloma, and
wherein the individual has renal impairment at the start of treatment.

4. The anti-CD38 antibody for use according to any one of claims 1-3, wherein the individual received 1-3 prior therapies for multiple myeloma, and wherein the treatment extends progression free survival (PFS) and/or overall survival (OS) of the individual.

5. The anti-CD38 antibody for use according to any one of claims 1-3, wherein the individual received 1-3 prior therapies for multiple myeloma.

6. The anti-CD38 antibody for use according to any one of claims 1-3, wherein the individual received more than three prior therapies for multiple myeloma.

7. The anti-CD38 antibody for use according to any one of claims 1-3, wherein the individual received prior therapy with a proteasome inhibitor.

8. The anti-CD38 antibody for use according to any one of claims 1-3, wherein the individual received prior therapy with an immunomodulatory agent.

9. The anti-CD38 antibody for use according to any one of claims 1-3, wherein the individual is classified as Stage I or Stage II according to the Revised International Staging System for multiple myeloma (R-ISS) at the start of treatment.

10. The anti-CD38 antibody for use according to any one of claims 1-3, wherein the individual is classified as Stage III according to R-ISS at the start of treatment.

11. The anti-CD38 antibody for use according to any one of claims 1-3, wherein the individual is not classified according to R-ISS at the start of treatment.

12. The anti-CD38 antibody for use according to any one of claims 1-3, wherein the individual has one or more cytogenetic abnormalities selected from the group consisting of: del(17p), t(4;14), and t(14;16).

13. The anti-CD38 antibody for use according to any one of claim 1 or 2, wherein the individual has renal impairment at the start of treatment.

14. The anti-CD38 antibody for use according to any one of claims 1-3, wherein the individual is 65 to less than 75 years of age at the start of treatment.

15. The anti-CD38 antibody for use according to any one of claims 1-3, wherein the individual is 75 years of age or older at the start of treatment.

16. The anti-CD38 antibody for use according to any one of claims 1-3, wherein the anti-CD38 antibody comprises a heavy chain variable region (V_{H}) comprising an amino acid sequence of SEQ ID NO: 7 and a light chain variable region (V_{L}) comprising an amino acid sequence of SEQ ID NO: 7 or SEQ ID NO: 9.

17. The anti-CD38 antibody for use according to any one of claims 1-3, wherein the anti-CD38 antibody is isatuximab.

18. The anti-CD38 antibody for use according to any one of claims 1-3, wherein the anti-CD38 antibody, the carfilzomib, and the dexamethasone are administered in a first 28-day cycle, wherein, the anti-CD38 antibody is administered at the dose of 10 mg/kg on Days 1, 8, 15, and 22 of the first 28-day cycle, the carfilzomib is administered at the dose of 20 mg/m² on Days 1 and 2 and at a dose of 56 mg/m² on Days 8, 9, 15, and 16 of the first 28-day cycle, and the dexamethasone is administered at the dose 20 mg on Days 1, 2, 8, 9, 15, 16, 22, and 23 of the first 28-day cycle.

19. The anti-CD38 antibody for use according to claim 18, wherein the anti-CD38 antibody, the carfilzomib, and the dexamethasone are further administered in one or more 28-day cycles following the first 28-day cycle,
wherein the anti-CD38 antibody is administered at the dose of 10 mg/kg on Days 1 and 15 of the one or more 28-day cycles following the first 28-day cycle, the carfilzomib is administered at a dose of 56 mg/m² on each of Days 1, 2, 8, 9, 15, and 16 of the one or more one or more 28-day cycles following the first 28-day cycle, and the dexamethasone is administered at the dose 20 mg on Days 1, 2, 8, 9, 15, 16, 22, and 23 of the one or more one or more 28-day cycles following the first 28-day cycle.

20. The anti-CD38 antibody for use according to any one of claim 1 or 3, wherein the individual is MRD negative at a threshold of 10⁻⁵ or less after treatment.

21. A method of treating a human individual having multiple myeloma, comprising administering to the individual an anti-CD38 antibody comprising (a) a heavy chain variable domain (V_{H}) that comprises: a CDR-H1 comprising the amino acid sequence DYWMQ (SEQ ID NO: 1), a CDR-H2 comprising the amino acid sequence TIYPGDGDTGYAQKFQG (SEQ ID NO: 2), and a CDR-H3 comprising the amino acid sequence GDYYGSNSLDY (SEQ ID NO: 3), and (b) a light chain variable domain (V_{L}) that comprises: a CDR-L1 comprising the amino acid sequence KASQDVSTVVA (SEQ ID NO: 4), a CDR-L2 comprising the amino acid sequence SASYRYI (SEQ ID NO: 5), and a CDR-L3 comprising the amino acid sequence QQHYSPPYT (SEQ ID NO: 6), carfilzomib, and dexamethasone,
wherein the anti-CD38 antibody is administered at a dose of 10 mg/kg, the carfilzomib is administered at a dose of 20 mg/m² or 56 mg/m², and the dexamethasone is administered at a dose of 20 mg,
wherein the individual received at least one prior therapy for multiple myeloma, and
wherein the treatment extends progression free survival (PFS) of the individual.

22. The method of claim 21, wherein the treatment extends overall survival (OS) of the individual.

23. A method of treating a human individual having multiple myeloma, comprising administering to the individual an anti-CD38 antibody comprising (a) a heavy chain variable domain (V_{H}) that comprises: a CDR-H1 comprising the amino acid sequence DYWMQ (SEQ ID NO: 1), a CDR-H2 comprising the amino acid sequence TIYPGDGDTGYAQKFQG (SEQ ID NO: 2), and a CDR-H3 comprising the amino acid sequence GDYYGSNSLDY (SEQ ID NO: 3), and (b) a light chain variable domain (V_{L}) that comprises: a CDR-L1 comprising the amino acid sequence KASQDVSTVVA (SEQ ID NO: 4), a CDR-L2 comprising the amino acid sequence SASYRYI (SEQ ID NO: 5), and a CDR-L3 comprising the amino acid sequence QQHYSPPYT (SEQ ID NO: 6), carfilzomib, and dexamethasone,
wherein the anti-CD38 antibody is administered at a dose of 10 mg/kg, the carfilzomib is administered at a dose of 20 mg/m² or 56 mg/m², and the dexamethasone is administered at a dose of 20 mg,
wherein the individual received at least one prior therapy for multiple myeloma, and
wherein the treatment extends overall survival (OS) of the individual.

24. A method of treating a human individual having multiple myeloma, comprising administering to the individual an anti-CD38 antibody comprising (a) a heavy chain variable domain (V_{H}) that comprises: a CDR-H1 comprising the amino acid sequence DYWMQ (SEQ ID NO: 1), a CDR-H2 comprising the amino acid sequence TIYPGDGDTGYAQKFQG (SEQ ID NO: 2), and a CDR-H3 comprising the amino acid sequence GDYYGSNSLDY (SEQ ID NO: 3), and (b) a light chain variable domain (V_{L}) that comprises: a CDR-L1 comprising the amino acid sequence KASQDVSTVVA (SEQ ID NO: 4), a CDR-L2 comprising the amino acid sequence SASYRYI (SEQ ID NO: 5), and a CDR-L3 comprising the amino acid sequence QQHYSPPYT (SEQ ID NO: 6), carfilzomib, and dexamethasone,
wherein the anti-CD38 antibody is administered at a dose of 10 mg/kg, the carfilzomib is administered at a dose of 20 mg/m² or 56 mg/m², and the dexamethasone is administered at a dose of 20 mg,
wherein the individual received at least one prior therapy for multiple myeloma, and
wherein the individual is Minimal Residual Disease negative at a threshold of 10⁻⁵ or less after treatment.


SEQUENCE LISTING (11 sequences)

SEQ ID NO: 1 - protein, 5 aa, Artificial Sequence
  Synthetic Construct
DYWMQ

SEQ ID NO: 2 - protein, 17 aa, Artificial Sequence
  Synthetic Construct
TIYPGDGDTGYAQKFQG

SEQ ID NO: 3 - protein, 11 aa, Artificial Sequence
  Synthetic Construct
GDYYGSNSLDY

SEQ ID NO: 4 - protein, 11 aa, Artificial Sequence
  Synthetic Construct
KASQDVSTVVA

SEQ ID NO: 5 - protein, 7 aa, Artificial Sequence
  Synthetic Construct
SASYRYI

SEQ ID NO: 6 - protein, 9 aa, Artificial Sequence
  Synthetic Construct
QQHYSPPYT

SEQ ID NO: 7 - protein, 120 aa, Artificial Sequence [sequence omitted]

SEQ ID NO: 8 - protein, 108 aa, Artificial Sequence
  Synthetic Construct
DIVMTQSHLSMSTSLGDPVSITCKASQDVSTVVAWYQQKPGQSPRRLIYSASYRYIGVPDRFTGSGAGTDFTFTISSVQAEDLAVYYCQQHYSPPYTFGGGTKLEIKR

SEQ ID NO: 9 - protein, 108 aa, Artificial Sequence
  Synthetic Construct
DIVMAQSHLSMSTSLGDPVSITCKASQDVSTVVAWYQQKPGQSPRRLIYSASYRYIGVPDRFTGSGAGTDFTFTISSVQAEDLAVYYCQQHYSPPYTFGGGTKLEIKR

SEQ ID NO: 10 - protein, 449 aa, Artificial Sequence
  Synthetic Construct
QVQLVQSGAEVAKPGTSVKLSCKASGYTFTDYWMQWVKQRPGQGLEWIGTIYPGDGDTGYAQKFQGKATLTADKSSKTVYMHLSSLASEDSAVYYCARGDYYGSNSLDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 11 - protein, 214 aa, Artificial Sequence
  Synthetic Construct
DIVMTQSHLSMSTSLGDPVSITCKASQDVSTVVAWYQQKPGQSPRRLIYSASYRYIGVPDRFTGSGAGTDFTFTISSVQAEDLAVYYCQQHYSPPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC